(19) United States
(12) Patent Application Publication
(10) Pub. No.: US 2021/0122667 A1
(43) Pub. Date: Apr. 29, 2021

(54) UV-C WAVELENGTH RADIALLY EMITTING PARTICLE-ENABLED OPTICAL FIBERS FOR MICROBIAL DISINFECTION

(71) Applicants: Paul K. Westerhoff, Scottsdale, AZ (US); Shahnawaz Sinha, Chandler, AZ (US); Sergio Garcia-Segura, Tempe, AZ (US); Mariana Lanzarini-Lopes, Tempe, AZ (US)

(72) Inventors: Paul K. Westerhoff, Scottsdale, AZ (US); Shahnawaz Sinha, Chandler, AZ (US); Sergio Garcia-Segura, Tempe, AZ (US); Mariana Lanzarini-Lopes, Tempe, AZ (US)

(21) Appl. No.: 17/137,790

(22) Filed: Dec. 30, 2020

Related U.S. Application Data
(63) Continuation-in-part of application No. PCT/US2019/053733, filed on Sep. 30, 2019.
(60) Provisional application No. 62/739,519, filed on Oct. 1, 2018.

(51) Int. Cl. C03C 13/04; C01B 33/12; C03C 25/105; C03C 25/106; G02B 6/10; F21V 8/00

(52) U.S. Cl. CPC C03C 13/045; C01B 33/12; C03C 25/105; B82Y 40/00; G02B 6/102; G02B 6/0073; C03C 25/106

(57) ABSTRACT

A coated optical fiber coupled to a light source for inactivating pathogens on surfaces or in water. The coated optical fiber includes a substantially UV-transparent core, particles optically coupled to the core, and a substantially UV-transparent polymer coating in contact with the particles. Coating the optical fiber includes optically coupling particles to a surface of an optical fiber core to yield a functionalized core, coating the functionalized core with a polymerizable material, and polymerizing the polymerizerable material to yield a substantially UV-transparent polymer coating on the functionalized core.

### CROSS-REFERENCE TO RELATED APPLICATION

This application claims the benefit of PCT Application No. PCT/US2019/053733, filed Sep. 30, 2019, which claims benefit of U.S. Provisional Application No. 62/739,519, filed Oct. 1, 2018, both of which are incorporated by reference herein in their entirety.

### STATEMENT OF GOVERNMENT INTEREST

This invention was made with government support under 1449500 awarded by National Science Foundation. The government has certain rights in the invention.

### BACKGROUND

Chemical oxidants produce potential harmful disinfection by-products (DBPs) and require on-site storage or production from liquid or gaseous feedstocks. Germicidal irradiation using light between 250 to 300 nm wavelengths (UV-C) does not produce DBPs and requires only electrical power. Low- and medium-pressure mercury lamps encased in quartz tubes are the most widely used UV-C light sources for water treatment. Multiple quartz tubes are usually installed in pipes or basins at water and wastewater treatment plants and disinfect water with less than one minute of contact time. Low-pressure mercury lamps are also employed in point-of-use, portable and industrial water purification systems. Light emitting diodes (LEDs) are becoming a competitive and lower cost alternative with promising characteristics for water treatment (e.g., lack of warm up time, tunable radiation, no degradation from on/off cycles and longer life of use). A crucial technology barrier of LEDs for disinfection is their small surface area that emits irradiation, which could require arrays of many LEDs in even the smallest reactors. However, it is impractical to use LED arrays or large quartz lamps in certain reactor geometries or other tight spaces where biofilms can grow (e.g., tubing, piping, storage tanks, ventilation ducts, medical devices, spiral-wound membranes and others). Thus, there is a need to distribute light from LEDs for microbial disinfection within reactors and for use in tight spaces to prevent biofilm formation.

### SUMMARY

Coated optical fibers described herein for delivery of UV light are small, compact, chemical-free, safe, and portable, and can be used to disinfect hard-to-reach areas, including in water and on surfaces, thereby reducing microbial and biofilm related risks. The coated optical fibers can be used in both as a fixed, standalone system or a portable disinfection unit.

In a first general aspect, a coated optical fiber includes a substantially UV-transparent core, particles optically coupled to the core, and a substantially UV-transparent polymer coating in contact with the particles. As used herein, a UV-transparent material is typically a UV-C transparent material. As used herein, a material that is “substantially UV-transparent” in a selected wavelength range typically has an average percent transmission of least 80%, at least 85%, at least 90%, or at least 95% over the selected wavelength range. For example, a material that is substantially UV-C transparent typically has an average percent transmission of at least 80%, at least 85%, at least 90%, or at least 95% over the UV-C wavelength range.

Implementations of the first general aspect may include one or more of the following features.

The particles may be adhered directly to the core surface or affixed proximate the core. The polymer coating may encase the UV-transparent core and the particles adhered to the core. The particles may include silica nanoparticles or silica beads (e.g., silica particle beads). The silica beads may be microspheres. The nano- or other particles are typically aminated to achieve a positive surface charge that is opposite in charge to the glass core negative surface charge, thus resulting in an electrostatic attraction between the core and nanoparticles. An average diameter of the particles is typically in a range from about 50 nm to about 500 nm (e.g., from about 200 nm to about 500 nm or from about 200 nm to about 400 nm). UV light passing through the core is scattered by the particles through the polymer coating. A thickness of the polymer coating is typically between about 10 μm and about 100 μm.

One approach includes optically coupling the particles to the surface of the core by integrating particles within an UV-transparent polymer. A thin layer (<1 μm) of UV-transparent polymer containing 0.5 wt % to 2 wt % of particles is deposited on the fiber, and then overcoated with a second layer of polymer devoid of particles. The particles may include silica nanoparticles or beads. The silica nanoparticles or beads may be amine functionalized, and typically have a diameter in a range of 50 nm to 500 nm.

Another approach involves modulating the distance, between 5 to 100 nm, between the core surface and the particles to tune the amount of light scattered from the fiber system by scattering refracted light at the interface of the fiber and control the amount of light energy interacting with the particles through evanescent wave interactions. The particles may include silica nanoparticles or beads. The silica nanoparticles or beads may be amine functionalized, and typically have a diameter in a range of 50 nm to 500 nm. The separation distance can be controlled by electrostatic interaction between the core surface charge (usually negatively charged) and particle surface charge (usually positively charged), plus ionic strength (at least 0.01M) of liquid used in adhering the particles.

In a second general aspect, coating an optical fiber having a core includes optically coupling particles to a surface of the core to yield a functionalized core, coating the functionalized core with a polymerizable material, and polymerizing the polymerizerable material to yield a polymer coating on the functionalized core, wherein the polymer coating is substantially UV transparent and flexible in nature.

In a third general aspect, a disinfectant system includes the coated optical fiber of the first general aspect.

In a fourth general aspect, an apparatus includes a light source coupled to the coated optical fiber of the first general aspect. The light source may include a light-emitting diode (LED) (e.g., a UV-C LED). In some cases, the light source is optically coupled to a heat sink.

### BRIEF DESCRIPTION OF THE DRAWINGS

FIG. 1A depicts an optical fiber with core, cladding, and coating, showing the light transmission, scattering and absorption through a single unmodified optical fiber. FIG. 1B depicts an optical fiber with core, cladding, and coating, showing the light transmission, scattering and absorption through an optical fiber modified to include scattering centers in the optical fiber cladding.

FIG. 2A depicts an unmodified optical fiber used to distribute light from a first end to a second end of the optical fiber. FIG. 2B depicts a modified optical fiber used to distribute light radially along the length of the optical fiber.

FIG. 3A depicts a tower for fabricating modified optical fiber and controlling particle addition into the cladding. As illustrated, two coating steps are used: the first (primary) deposits the modified cladding (cladding polymer+particles) onto the fiber. The second deposits the outer protective polymer. FIG. 3B depicts an optical setup for light attenuation and scattering analysis. P1 and P2 represent the sensor positions 1 and 2, respectively.

FIGS. 4A-4D are optical microscope images at 40× magnification of optical fibers of: 0 wt % (neat-clad), 0.5 wt % SiO₂-clad, 1.0 wt % SiO₂-clad, and 2.0 wt % SiO₂-clad, respectively.

FIGS. 5A and 5B depict inducing Mie scattering at the surface of the core of an optical fiber to allow for increased light distribution by adding silica directly to the optical fiber and further adding a transparent coating to maintain the strength and flexibility of the optical fiber.

FIG. 6A shows the effect of sulfate (and ionic strength) on increased light scattering through the optical fiber. FIG. 6B shows scattered flux with and without silica particles and with and without a polymer coating.

FIG. 7 depicts an optical set-up for light attenuation and scattering analysis.

FIG. 8A shows the effect of optical fiber clad functionalization on the relative scattered flux at 1 m position (I_{S,1m}) for a deuterium lamp source input (I₀) (λ=350-570 nm). The inset represents the relative scattered flux optical fiber cladding functionalized with 1.0 wt % by BaSO₄ and ZONYL. FIG. 8B shows the effect of optical fiber clad functionalization with SiO₂ spheres on the attenuation of light through the optical fiber for 2=350-570 nm. The inset represents the functionalization of the fiber clad with 1.0 wt % BaSO₄ and ZONYL.

FIG. 9 shows logarithmic reduction in scattered flux along the length (position 0 to 2.5 m) of the 2.0 wt % SiO₂ optical fiber.

FIG. 10 shows scattered flux increases from optical fibers coated with silica spheres with a 265 nm UV-C LED (10 mW/cm²).

FIG. 11 shows a process used to fabricate UV-C scattering optical fibers.

FIG. 12 depicts an optical fiber illustrating that photons that are coupled into an optical fiber (I₀) can be transmitted (Iₜ) through the optical fiber core by internal reflection, absorbed (Iₐ) by the core or cladding materials, or side emitted (Iₛ) by scattering of the optical fiber.

FIG. 13 depicts a potassium ferrioxalate actinometry experimental set up used to obtain total scattering from the test fiber.

FIG. 14 shows theoretical scattering and absorption cross sectional area of gold, silver, and silica predicted based on Oldenburg's Mie theory calculator. The extinction coefficient is the amount of light a particle removes from a beam normalized by the geometric cross sectional area of the particle that is normal to the beam. Here, the light is either absorbed (absorption cross sectional area) or scattered (scattering cross sectional area). The silica spheres illustrated negligible absorption for both 50 nm and 500 nm diameters and significant scattering for the 500 nm spheres.

FIG. 15 shows absorbance of silica spheres of different diameters at 265 nm wavelength by diffuse reflective spectroscopy corrected by the Kubelka-Munk equation. Values illustrated are averaged triplicates with one standard deviation above and below. A schematic of increased absorption as the particle size decrease is illustrated in the inset.

FIG. 16 shows the effect of the particle size and loading on 265 nm wavelength-localized scattering flux. Particle loading was varied by the number of dip-coating cycles. Each dipping cycle resulted in (0.41 μg/mm²±6%) additional loading for all of the sizes. Localized scattering flux was measured by a spectrophotoradiometer at the midpoint of the optical fiber (2.5 cm), as illustrated in FIG. 11. Values illustrated are averaged triplicates with one standard deviation above and below.

FIG. 17 shows a scanning electron microscope (SEM) image of optical fiber cross section after the four-step preparation depicted in FIG. 11.

FIG. 18 shows localized scattering flux (Iₛ) of 265 nm for optical fiber coated with 400 nm silica increased with high ionic strength treatment. The ionic strength was controlled by increasing concentrations of Na₂SO₄ (solid square) and Na₃PO₄ (solid diamond). Values illustrated for Na₂SO₄ are averaged triplicates with one standard deviation above and below.

FIG. 19 shows localized scattering flux (Iₛ) of 265 nm for optical fiber after preparation Steps 1, 2, and 3 without CYTOP (open bars) and with CYTOP (solid bars). Increase in localized scattering from each step is not affected by the CYTOP coating. The inset illustrates absorption of 265 nm wavelength by CYTOP, PMMA and DESOLITE 0016 polymers as measured by DRS. The polymer thickness between 5 and 20 μm was deposited on the quartz substrate and measured for each sample. Absorbance was adjusted to 5 μm by the Beer-Lambert law. Values illustrated are averaged triplicates with one standard deviation above and below.

FIG. 20 shows log inactivation of E. coli by coupling a UV 265 nm wavelength LED to a side-emitting optical fiber (solid triangles) that included all four preparation steps and a control (solid squares) that included only Steps 1 and 4 (i.e., a clean fiber coated with CYTOP). The control illustrates that the CYTOP did not contribute E. coli inactivation. The illuminated optical fiber was submersed in a 5 ml solution of E. coli, and log inactivation of triplicate samples were enumerated in duplicates. All samples were adjusted to a dark control to account for any growth or inactivation due to environmental conditions.

FIG. 21 shows the distribution of light through the length of the four-step modified optical fiber. Inlet, transmitted, and scattered light at 5 cm was measured by chemical actinometry and the absorbed light was calculated with Eq 1. The amount of light transmitted at each length is calculated by the Beer-Lambert law where α=0.76 dB/cm. By assuming the fiber length=∞(T=0), the amount of inlet light scattered (desired) versus absorbed (not desired) was compared. The scattering ratio Is/(Is+Ia) was calculated to be 85% for the four-step modified optical fiber and ˜1% for the unmodified optical fiber (not shown). The scattering ratio then predicts the total fraction of inlet 56 light that is scattered and absorbed through the fiber length.

FIG. 22A is an exploded view of a device including a UV-C LED coupled to a UV-C side-emitting optical fiber. FIG. 22B is an assembled view of the device in FIG. 22A.

FIGS. 23A-23E show UV-C light irradiance measured at different distances along an optical fiber with 0.02M Na₂SO₄, 0.05M Na₂SO₄, 0.10M Na₂SO₄, 0.15M Na₂SO₄, and 0.20M Na₂SO₄, respectively. FIG. 23F shows linear fitting of scattering coefficient (left) and integrated light intensity (right) with the concentration of Na₂SO₄ used on the side emitting optical fiber (SEOF). The standard deviations for triplicate independent optical fibers are illustrated as error bars.

FIGS. 24A and 24B show schematically how ionic strength impacts separation distances between aminated silica nanoparticles and am optical fiber glass surface, thus increasing the light scattering through greater interaction with the evanescent wave energy.

FIG. 25 shows scattering flux along 30 cm optical fiber treated with a series of high ionic strength solutions. A dip-coating method was applied to process different positions along the optical fiber. The fiber at 6 cm, 12 cm, 18 cm and 24 cm was submerged into 0.02M, 0.05M, 0.10M, and 0.15M sodium sulfate solution, respectively. The standard deviation for triplicate independent optical fibers are illustrated as error bars.

FIGS. 26A and 26B show zones of inhibition for P. aeruginosa and E. coli, respectively, through the length of the optical fiber. FIG. 26C shows zones of inhibition as an average of the fiber length. Both organisms were exposed to UV radiation for 0, 30, 60, 120, 240 and 480 minutes. A dark control was analyzed by inserting the SiO₂ modified optical fiber in the agar plate with no UV exposure. The error bars represent one standard deviation between independent experiments.

### DETAILED DESCRIPTION

Coated optical fibers suitable for radial, side-emission delivery of UV (e.g., UV-C) light are described. As used herein, “UV light” typically refers to UV-C light. The coated optical fibers are fabricated by combining particles (e.g., particle beads, such as silica beads) of a selected size (e.g., diameter) to a flexible, transparent core (e.g., glass) in a cladding on the core, and coating the cladding with a polymer. The size of the particles is selected based at least in part upon the light wavelength being employed to achieve a desired scattering of the UV light. The polymer is typically a UV (e.g., UV-C) transparent polymer coating that can enhance flexibility and durability of the optical fibers. The polymer may be applied by dip coating, spray coating, extrusion, surface polymerization or other processes. Light from a light source (e.g., LED, mercury lamp, laser) is launched into a single coated optical fiber or a bundle of the coated optical fibers and radially emitted along the length of the fiber(s). The mass loading of particles on the fiber affects the amount of UV light emitted per unit length of the optical fiber. The surface density of particles (μg/cm²) adhered on or near the fiber core surface may be constant with the length of the fiber, or varied with the length of the fiber to control the amount of side emitted light from the fiber with distance away from the light source. More uniform side-emitted light can be achieved by varying, or tapering, the particle surface density with length of the fiber.

The coated optical fibers can be placed in a vessel containing a fluid and used to deliver light (e.g., UV-C) to disinfect the vessel and its contents (e.g., by controlling biofilms, inactivating planktonic microorganisms, inactivating airborne microorganisms and the like). The coated optical fibers can be used for treating drinking water or air ventilation or medical devices or in industrial processing or military or space applications.

Light (W/cm²) launched into an optical fiber (I₀) fiber can be transmitted (I_{T}), absorbed (I_{A}), or scattered (I_{S}) as it travels its length (L (m)) such that summation of terms as shown in Eq. (1) accounts for all the light:

I₀=I_{T}+I_{A}+I_{S} (1)

Attenuation

$$( α ( \frac{a {B}_{T}}{m} ) )$$

through the optical fiber, described by the Beer-Lambert law Eq. (2), relates I_{T} to the properties of the material that it is traveling through.

$$α = \frac{- 10 \cdot {log}_{10} ( \frac{{I}_{T}}{{I}_{0}} )}{L} . \qquad ( 2 )$$

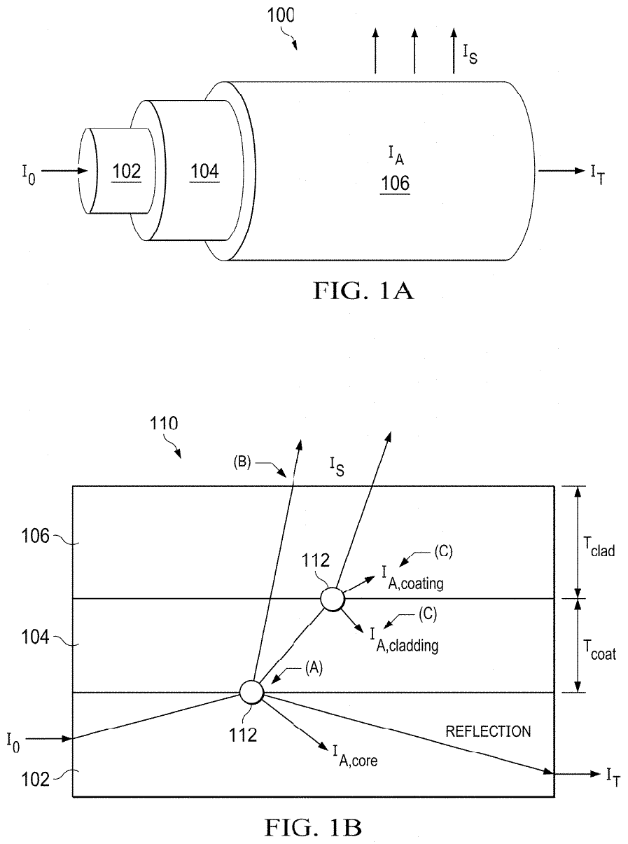

FIG. 1A depicts light transmission (I_{T}), scattering (I_{S}), and absorption (I_{A}) through a single unmodified optical fiber 100 having a core 102, a cladding 104, and a coating 106. This optical fiber has a light guiding core 102 that minimizes the light scattering or light leakage or bleeding from its outer walls by having two protective polymer layers (i.e., cladding 104 and coating 106). These outer layers assure both strength and the flexibility for uniform light passage and transport with minimal loss through core 102.

FIG. 1B shows an optical fiber 110 with a layer 104 that includes scattering centers 112 (e.g., particles, such nanoparticles or beads) and a polymer coating 106, with light scattering out of the optical fiber core 102, absorption by the polymer coating 106, and scattered photon flux. Scattering centers 112 in direct contact with core 102 directly influences the attenuation through the optical fiber 110 by scattering light away from the guiding core 102 (see (A) in FIG. 1B). Scattering centers 112 are typically present in an amount of about 0.5 wt % to about 5 wt % of polymer coating 106. Scattering centers 112 cause light to either exit the fiber 110 (see (B) in FIG. 1B) and be measured as I_{S} or be absorbed by the layer 104 or coating 106 (see (C) in FIG. 1B). Attenuation can be quantified, for example, by the cutback method, where the fiber 110 is cut back 3.0 m from 5.0 m long to 2.0 m long. Eq. (2) can then be used to calculate a by measuring the transmittance at both 5.0 m (I_{T}) and 2.0 m (I₀). The scattered flux at a specific length (I_{S,L}) can be normalized by I₀ and reported as the fractional scattered flux (I_{S,L}/I₀).

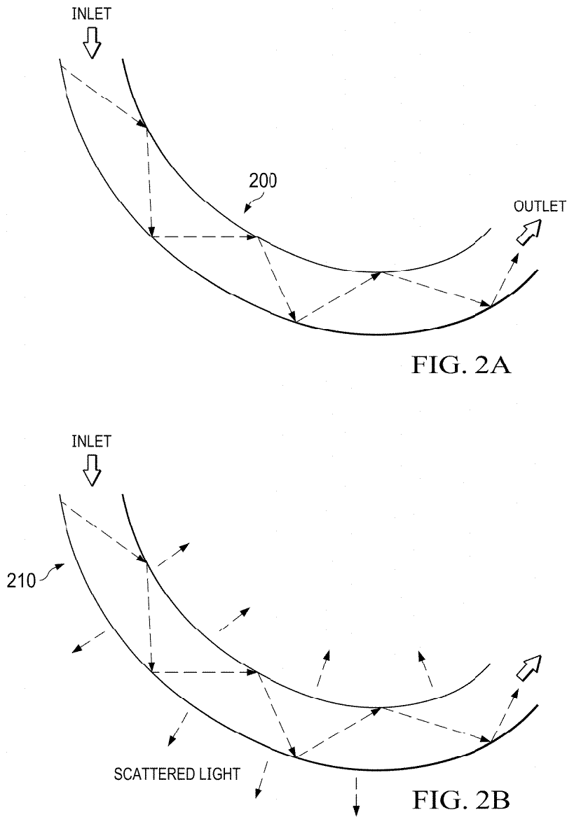

FIG. 2A depicts internal reflection of a convention optical fiber 200. FIG. 2B depicts an optical fiber 210 with scattering centers (not shown) that allow passage of UV light through the coating of the optical fiber.

Suitable scattering centers can be selected to span a range of particles, such as barium sulfate, a white crystalline polytetrafluorethylene powder (ZONYL), and silica microspheres. Experiments with barium sulfate and ZONYL showed a small increase in radial scattering compared to that of the unmodified optical fiber (UV=1.3× and visible=7.0× for barium sulfate and UV=0.4× and visible=6.0× for ZONYL). Incorporation of 500-nm SiO₂ particles showed increases of UV=9.7× and visible=95.2×. The impact of the scattering centers on a broad spectrum (UVA to visible light) was observed.

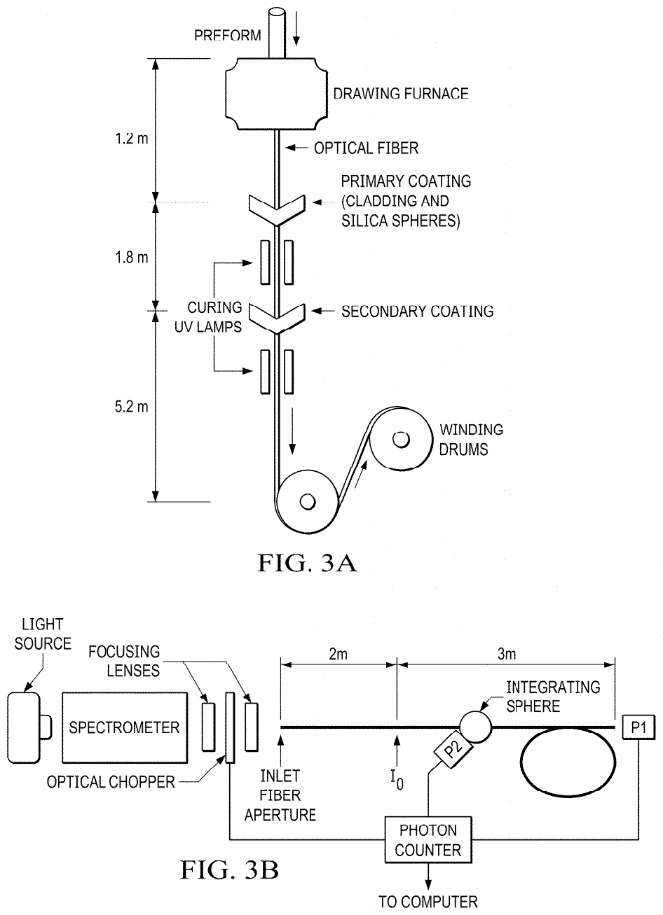

Custom optical fibers were manufactured at Lawrence Livermore National Laboratory in an 8.2 m tall draw tower. As illustrated in FIG. 3, fused silica glass rods F300 (Haraeus Gmbh) with 26 mm outer diameter were used as the raw material (preform) to draw 250-μm optical fibers. Briefly, the preform was placed in a 1,900° C. drawing furnace and dropped three stories through two sets of polymer dies (primary coating (cladding) and secondary coating) at 1.2 and 3.0 meters distance from the drawing furnace before being wound into 0.2 m diameter drums.

Polymer solutions were degassed in a low vacuum before connecting them to the die to avoid the formation of gas bubbles in the polymer. The nominal thickness of the applied polymer on the optical fiber is the difference between the die size and the fiber diameter divided by two. A 330 μm die was used to apply the 40 μm thick polymer cladding (DESOLITE DF-0016, DSM Desotech Inc.) followed by a 480 μm die to apply the 75 μm thick fiber secondary polymer coating (DESOLITE DS-2015, DSM Desotech Inc). Each polymer was UV cured at 387 nm after its application, causing the material to shrink slightly.

To induce scattering of light from the optical fiber core, functionalization of the cladding was tuned by loading silica microspheres (500-nm, Sigma-Aldrich: 805890) in the polymer at 0.5 wt %, 1.0 wt %, and 2.0 wt %. The 500 nm particle diameter was chosen to induce equal scattering along all wavelengths (Mie scattering). The microspheres were added to the DESOLITE DF-0016 (cladding polymer) and rapidly hand mixed for 20 minutes. The mixture was sonicated in a water bath for 4 hours and hand stirred for 10 additional minutes. The solution was again degassed in a low vacuum before connecting it to the primary coating die. An unmodified external polymer coating was applied to all of the optical fibers for additional protection.

A reference optical fiber with no modifications to the cladding (neat-clad) was fabricated and analyzed for comparison. Physical characterization of the optical fiber was obtained through 40× magnification microscope images through reflection mode (Leica DM6 B).

FIG. 3B illustrates the optical analysis setup used for both measurements. Light from a deuterium lamp (Newport, Q Series 30W) was directed though a spectrometer (HORIBA Jobin Yvon microHR) allowing monochromatic light analysis of selected wavelengths between 350 and 570 nm. Two planar-convex lenses (Thorlabs LA4148) were used to further couple the light into the optical fiber. An optical chopper (Thorlabs MC2000B controller and MC1F15 blade) reduced the signal noise by communicating a frequency signal to the photon counter detector (Model SR830 DSp Lock-in Amplifier Stanford Research Systems). The sensor was a bialkali photocathode coupled to a silica glass window photomultiplier tube (PMT) (Hamamatsu R760). I_{S,L} was measured by guiding the optical fiber through a 50.8 mm diameter integrating sphere (Thorlabs IS200-4). The integrating sphere is a hollow spherical cavity covered with a diffuse white reflective coating that allows all of the light scattered from the optical fiber to be captured and quantified.

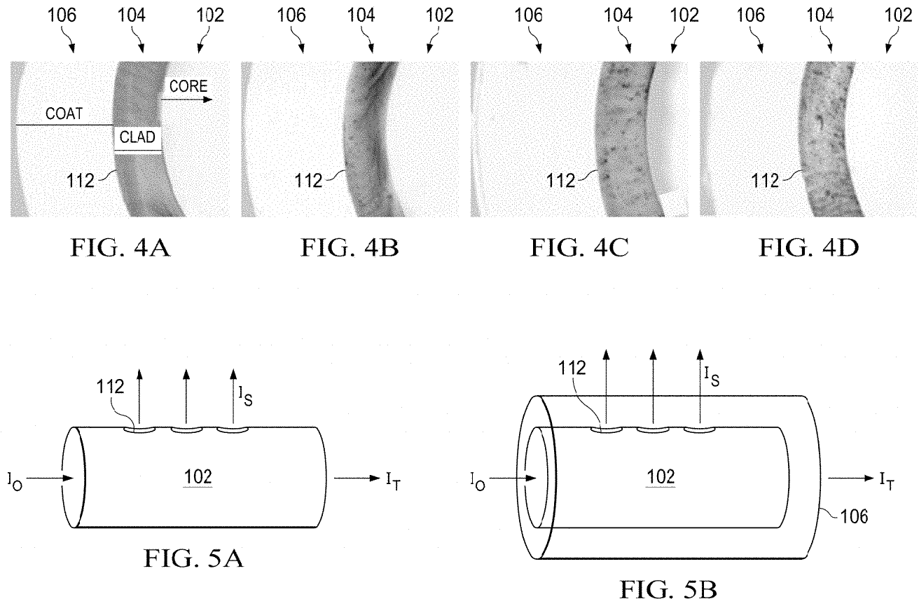

FIG. 4A is an optical microscope image showing the core 102, cladding 104, and coating 106 of a neat-clad optical fiber circular face. FIGS. 4B-4D are optical microscope images showing the core 102, cladding 104, and coating 106, with 0.5 wt %, 1.0 wt %, and 2.0 wt % loading, respectively, of 500 nm silica spheres in the cladding. This is done by adding particle beads (e.g., silica beads) directly to the optical fiber as a cladding material with a transparent polymer coating to promote more photons to escape or pass through as shown in FIGS. 5A and 5B. The particles 112 were not only present at the cladding-core interface but appeared well distributed throughout the thickness of the cladding. The thickness of the polymer cladding and coating after the curing process are 31.3 μm (±5.3%) and 62.8 μm (±4.8%), respectively. The polymer cladding and coating protects and strengthens the optical fiber, allowing it to bend without breaking (higher flexibility). To increase the scattered flux, a higher density of homogenously dispersed particles can be used to increase photon-particle interaction that results in scattering. However, loadings >2.0 wt % typically resulted in brittle coated fibers.

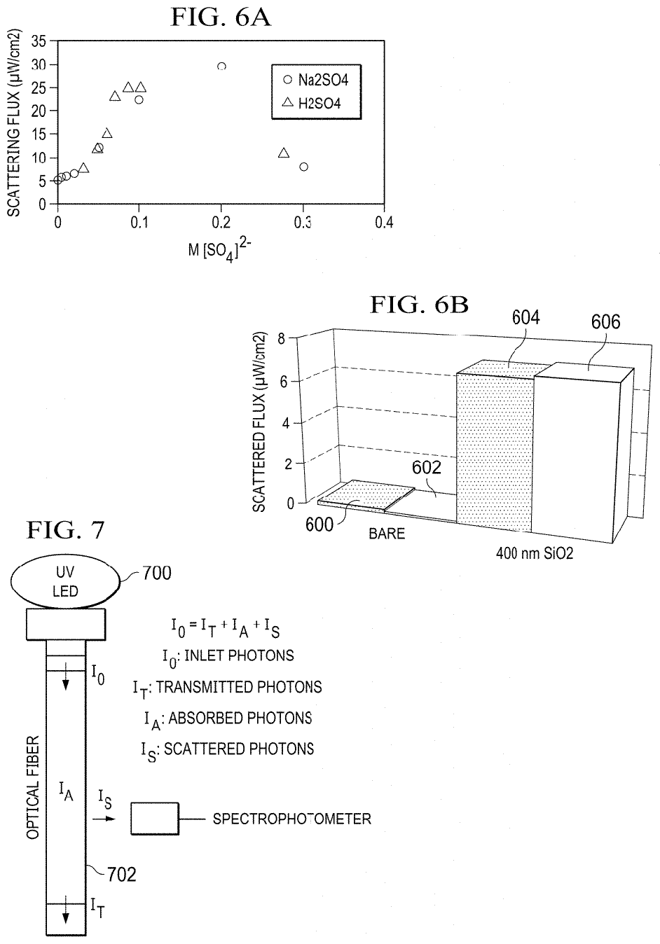

FIG. 6A shows higher sulfate concentration leads to increased light scattering. Moreover, a UV-C transparent polymer coating helps to maintain fiber strength and flexibility while providing light passage and scattering. Suitable polymers include CYTOP and poly(methyl methacrylate) (PMMA). Otherwise, the cladding will absorb UV light (e.g., UV-C light) and prevent its passage through the cladding and out of the fiber. FIG. 6B shows scattered flux an optical fiber with no scattering centers with and without a polymer coating in bars 600 and 602, respectively, and for an optical fiber with scattering centers (400 nm silica particles) with and without a polymer coating in bars 604 and 606, respectively.

As depicted in FIG. 7, a UV-LED (UV-C) based light source 700 can be used to launch UV light (e.g., UV-C light) into an end of an optical fiber 702 (single fiber or bundle). The UV-C light passes along the optical fiber 702. Light is scattered at the fiber surface due to the presence of the particles in the cladding (e.g., as illustrated in FIG. 5B. The loading and composition of the particles influences light scattering by the fibers.

Scattering flux through optical fibers can be increased by (i) increasing the scattering opportunities and (ii) decreasing absorption of light. Both the cladding and coating are highly absorbing in the UV region. The presence of scattering centers on the interface between the optical fiber core and cladding can increase the scattering opportunities as well as partially replace a highly absorbing cladding polymer.

There are two dominant types of linear light scattering in optical fibers: Mie and Raleigh scattering. Rayleigh scattering refers to the elastic scattering of light from a particle with a diameter (D) of about one-tenth the size of the incident wavelength (πD/λ<<1). Rayleigh scattering is defined by Eq. (3) where R is the distance of the scattering object from the detector, η is the number of scattering objects, λ is the wavelength of the propagated light, and θ is the scattering angle. Rayleigh scattering is mostly due to inherent uniformities within the optical fiber's core molecular structure and increases as 1/λ⁴.

$${I}_{S} = {I}_{0} \frac{1 + {cos}^{2} θ}{2 {R}^{2}} {( \frac{2 π}{λ} )}^{4} {( \frac{{n}^{2} - 1}{{n}^{2} + 1} )}^{2} {( \frac{D}{2} )}^{6} \qquad ( 3 )$$

Mie scattering occurs when the deformity is comparable to the size (D>λ/10) of the incident wavelength. Mie scattering is identified by the ability of particles to scatter all wavelengths of white light equally. The constant scattering in the visible range with a decrease in scattering in the UV region (where polymer illustrates high absorption) is therefore likely due to the absorption of light by the material.

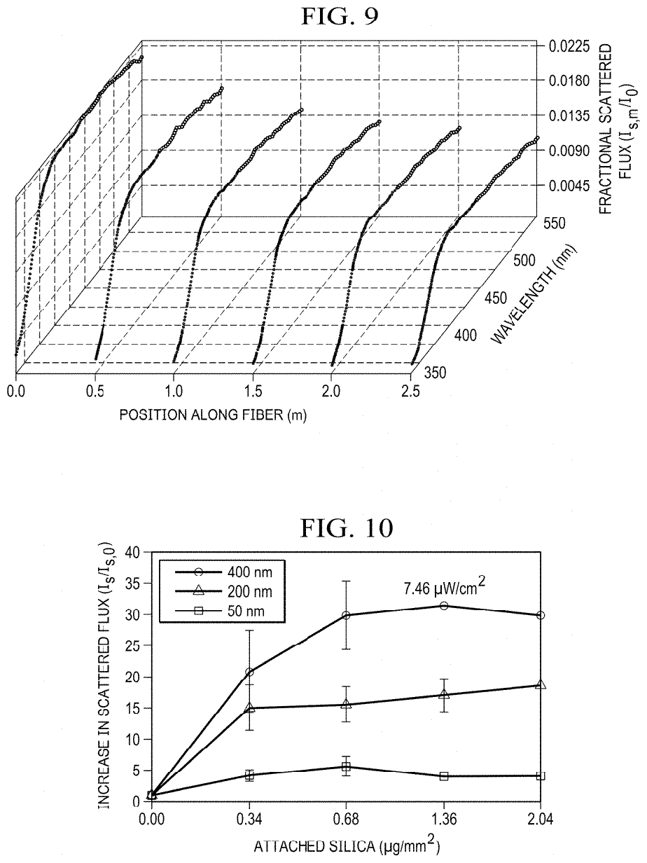

Mie theory indicates that I_{A}/I_{S} increases with decreasing diameter because the particle scattering cross sectional area decreases in proportion to its volume for d≤λ. Experimental results illustrate that scattering centers in the form of silica spheres with an average diameter exceeding 200 nm scattered more than scattering centers in the form of silica spheres with an average diameter below 200 nm. Further increasing the diameter does not statistically (student t-test) impact the result. FIG. 9A shows the effect of incorporating 500 nm silica spheres within the cladding on the fractional scattered flux. Scattering increased for all wavelengths as silica particle loading on the fiber increased from 0.5 wt % to 2.0 wt %. Rayleigh scattering was illustrated in the 0 wt % optical fiber where scattering increased as wavelength decreased. The equal increase of visible light scattering upon the addition of 500-nm silica spheres suggests that Mie scattering is the primary mechanism of increased scattering flux for the modified optical fiber.

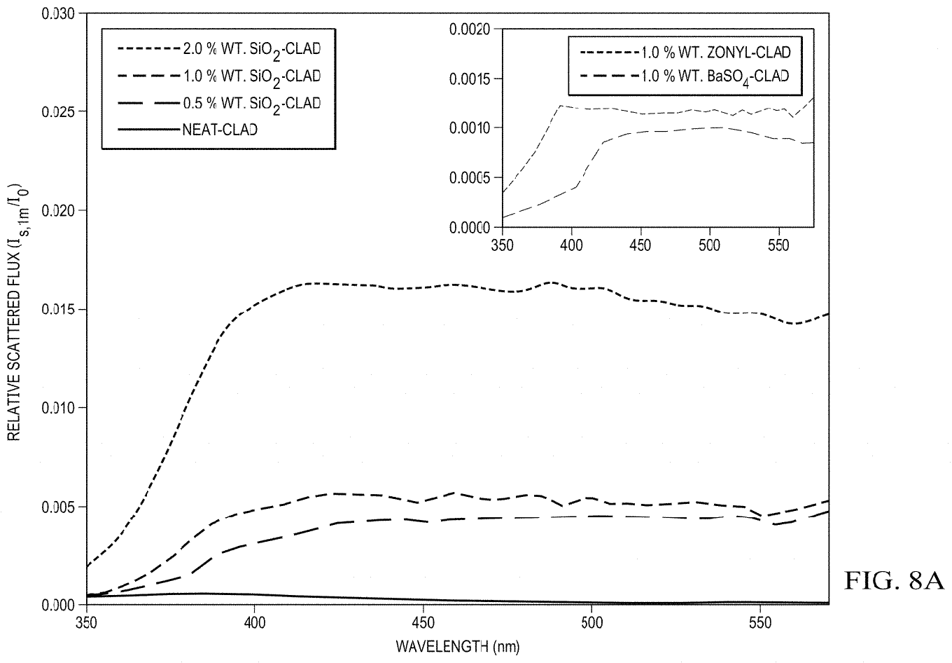

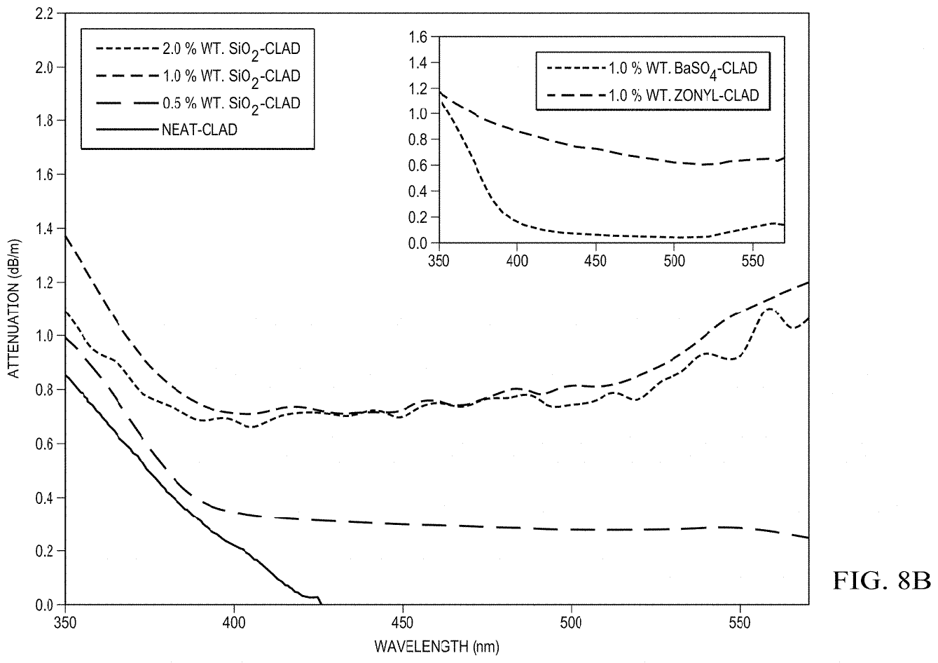

The scattering flux tripled when the SiO₂ loading increased from 1.0 wt % to the 2.0 wt %. As shown in FIG. 8A, the 2.0 wt % SiO₂-clad increased the scattered flux ratio by 9.7× to 30.3× in the UVA range and 95.2× for visible light when compared to the neat-clad fiber. This increase in scattering was due to a higher photon to particle interaction and to the replacement of the polymer cladding, which decreased absorption by the polymer.

FIG. 8A shows that the light scattering increases with increasing wavelength (350-550 nm). It also shows the positive effect of silica addition (i.e., loading) and functionalization (0.5 wt % to 2 wt %) of the fibers to the relative scattered flux and its increase. However, for the neat-clad optical fiber, this figure also shows that Raleigh scattering dominates without much scattering (where the scattering of light is due to silica core and/or polymer cladding and/or coating adsorption, without showing any effect of wavelength) with a UV-tail.

To investigate how attenuation through the fiber affects scattering distribution for both UVA and visible light, the scattered flux was measured at different positions along the fiber (0.0 m, 0.5 m, 1.0 m, 1.5 m, 2.0 m, and 2.5 m). FIG. 9 shows the relationship between fractional scattered flux (from 315 nm to 570 nm) and position along the fiber for the 2.0 wt % SiO₂-clad optical fiber. A logarithmic decay in scattered light is observed along the optical fiber. This observation is consistent with the Beer-Lambert law represented in Eq. (2) for every wavelength with a total decrease in scattering intensity of 46%±4.3% from optical fiber position 0.0 m to 2.5 m. This even decrease throughout the UVA and visible ranges indicates that the silica core did not significantly absorb more UVA than visible light when compared to the polymer cladding and coating. Most of the photons in the UVA region are absorbed by the polymer cladding and coating before they are measured as I_{S}.

To investigate the total loss through each optical fiber, the attenuation through the optical fiber was measured by the cutback method. FIG. 8B shows the effect of scattering center functionalization on the total attenuation. Light attenuation through the fiber is due at least in part to (i) absorption by the glass fiber or polymer cladding/coating, (ii) inherent scattering losses (Raleigh), and (iii) introduced Mie scattering due to silica loading onto the cladding of the optical fibers. Increased attenuation also decreases the useful length of optical fiber, where I_{T}/I₀>90%. The right axis of FIG. 8B represents the respective length for which 90% of the inlet light would be lost either due to scattering or absorbance of photons. The neat-clad fiber shows attenuation below the limit of detection (I_{T}/I₀>0.99) for the 3 m cutback length of the fiber. Addition of 0.5 wt % or 1.0 wt % SiO₂-clad optical fibers increased the total attenuation by 0.3 and 0.7 dB/m, respectively, in the visible range. The silica spheres likely disturbed the photon path along the fiber, increasing loses due to Mie scattering. Addition of 2.0 wt % silica did not further increase attenuation as the photon disturbance at the core/cladding interface reached a maximum. This due at least in part to internal reflection back through the optical fiber core, thus decreasing the attenuation. An increase in attenuation also indicates a decrease in useful length of the optical fiber, to which 90% of the inlet light would be lost either due to scattering or due to absorbance of photons. These results suggest a decrease in light scattering or increase in attenuation at (<400 nm) for the coated optical fiber. It is due at least in part to the absorbance of light by the silica core and/or polymer coating, thus silica sphere loading, polymer addition, etc. are factors in fabricating effective light scattering fibers while maintaining strength and flexibility for disinfection.

FIG. 10 shows the scattered flux under UV-C (265 nm; under 10 mW/cm² intensity) light with attached silica (μg/mm²) spheres on the surface of the optical fiber, but of different silica sphere sizes ranging from 50 nm to 400 nm to increase the overall scattering. This figure also suggests a significant increase in scattering is possible by the size of the silica sphere used. The neat fiber without silica loading is shown with low light scattering of about 0.3 μW/cm², whereas with larger silica spheres, the scattering could reach as high as 7.8 μW/cm². These results show effects of adding these impurities to fibers and sphere size selection.

Whereas visible light scattering uniformly increased with silica sphere addition, a different pattern was observed for UVA wavelengths (i.e., <400 nm). The neat-clad fiber fractional scattered flux increased with decreasing wavelength (FIG. 8A) for λ>385 nm following Rayleigh scattering trend (previously described). For λ<385 nm, a decreased trend of fractional scattered flux is illustrated for decreasing wavelength. This trend is a result of increased UV absorption by the polymer cladding and coating. FIGS. 8A and 8B show exponential decrease in scattering and increase in attenuation, respectively, for λ<400 nm light with and without addition of silica spheres. These results are due at least in part to absorbance by both the silica core and polymer cladding/coating. Therefore, to fabricate optical fibers that scatter light <400 nm, UV-light transparent polymers would be advantageous.

Thus, loading optical fiber cladding with scattering centers was an effective method to increase the scattered flux ratio in optical fibers by 9.7 to 30.3× in the UVA range and >95.2× in the visible range. This allows light to exit the fibers for a wide variety of potential applications. Existing optical fiber fabrication infrastructure can be used to produce evenly distributed scattering centers within the optical fiber polymer cladding. Higher concentrations of scattering centers led to a higher scattering flux ratio due to increased photon-particle interaction. The scattering flux was dominated by Rayleigh scattering for the bare optical fiber and Mie scattering for the optical fibers modified with silica spheres.

The increased absorption by the polymer cladding and coating with shorter wavelengths limits the scattering emission of UVA light from these optical fibers. Replacing the materials with UV-transparent (e.g., UV-C transparent) polymers and silica core would allow for higher scattering by decreasing loss (absorption) within the fiber. Scattering of UVB and UV-C wavelength ranges (254-365 nm) is suitable for use in water treatment applications. UV-scattering optical fibers and coupled with UV-LEDs can increase energy efficiency in applications such as UV disinfection (UV-C range) and oxidation of organics (UVA range).

Continuous side-emitting optical fibers can serve as a high surface area, LED light delivery technology for microbial disinfection within reactors or tubing. As described herein, the surfaces of conventional optical fibers can be modified with scattering centers to allow side emission of 265 nm radiation from an LED for microbial inactivation in water. Solid-material absorbance and light fluxes in water using a radiometer or chemical actinometry differentiated light absorption from scattering and guided selection of both scattering centers and polymer cladding material for the optical fiber surfaces. Silica spheres >200 nm in diameter achieved higher scattering than smaller diameter silica spheres. Adding a high ionic strength solution after attaching silica spheres to the glass optical fibers can increase UV-C radial emission by >6×. Additionally, UV-C transparent polymer cladding was selected to prevent release of the scattering center particles into the water and to protect the fiber and ensure mechanical integrity. The cladding polymer CYTOP had negligible absorbance at 5 μm thickness in comparison with other polymers (DESOLITE 0016 and PMMA). A scalable four-step treatment process was developed to fabricate side-emitting optical fibers. Attached to a 265 nm LED light source, the side-emitting optical fiber achieved 2.9 log inactivation of E. coli at a delivery dose of 58 mJ/cm². The results demonstrate that UV-C continuous side-emitting optical fibers can deliver LED light into water and thereby inactivate microorganisms in the water.

As depicted in FIG. 1A, optical fibers usually have a circular core made of glass or polymer that is encased by one or more coatings (e.g., cladding and coating). Traditional optical fibers propagate light axially (transmission) due to complete internal reflection from the lower refractive index cladding surrounding the higher refractive index core. Thin glass fibers can be remarkably flexible, but scratches or other damage initiate mechanical failure. The external secondary polymeric coatings protect the glass from breaking when bent. In some applications, only one layer is applied, serving as both the cladding and coating. Commercial-scale optical fiber production is conducted in multi-story drop towers, where glass is melted, molded and coated through a series of in-line rollers, polymer paths and heating or curing chambers. This produces optical fibers up to kilometer lengths.

Side- or radially-emitting optical fibers deviate from traditional light guides used in telecommunications and laser applications where scattering outside the fiber is undesirable. A barrier to emitting UV-C radiation from the side of the optical fibers is the absorption of light by the core and coating of the fiber. Modification of optical fibers to allow side-emission of UV-C light allows delivery of UV-C light for microbial inactivation in water treatment reactors with unique geometries.

As described herein, particles (“scattering centers”) were selected and attached to the surface of glass optical fiber cores. The scattering centers induce side-emission of UV-C light generated by a 265 nm LED and thus can inactivate E. coli in water. A scalable treatment process was developed to fabricate the optical fiber. The process includes selecting a UV-C transparent polymer cladding to minimize absorption of UV-C radiation by the fiber itself. Solid-material absorbance, radiometer, and chemical actinometry measurements were used to quantify adsorption versus scattering and to parameterize a design model for optical fibers.

### Fabrication and Experimental Methods

Fabrication of UV-C side-emitting optical fibers. Multimode, UV-transmitting optical fibers of 1 mm diameter, numerical aperture of 0.39, and core refractive index (RI) of 1.46 were purchased from THOR labs (FT10000UMT). These hard fluoropolymer clad silica fibers where chosen due at least in part to their low UV absorption by the silica core and ability to remove the coating and cladding.

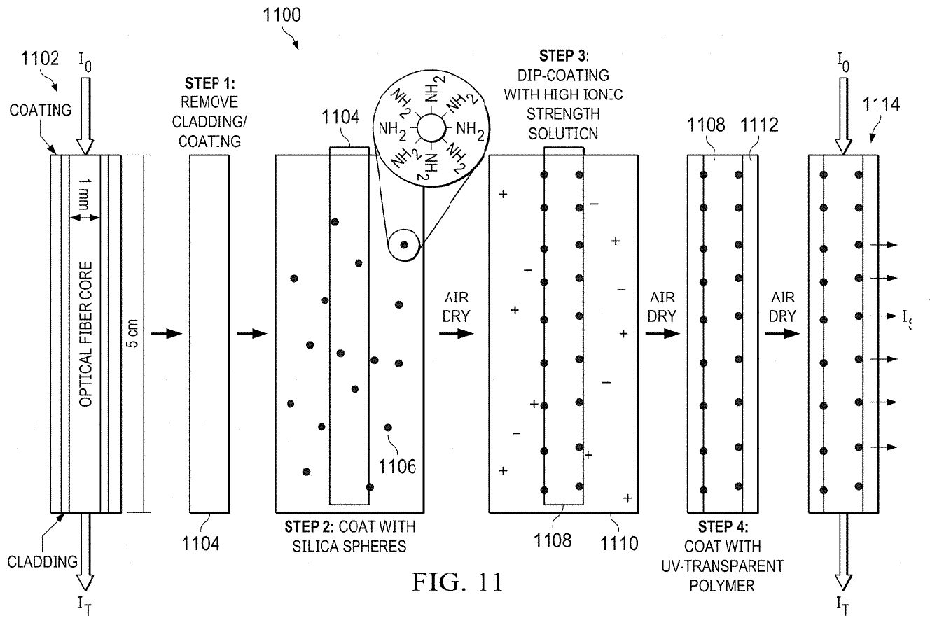

A fiber preparation process 1100 is depicted in FIG. 11. Process 1100 includes steps (1)-(4). Step (1) includes physical stripping of the cladding and coating polymers followed by an acetone bath to dissolve remaining cladding. Step (2) includes coating with silica spheres by dipping the optical fiber in aminated silica sphere ethanol suspension. Step (3) includes a high ionic strength treatment by dipping the optical fiber in a solution of Na₂SO₄. Step (4) includes dip-coating the optical fiber with CYTOP, a UV-transparent (e.g., UV-C transparent) polymer, for example at a rate of 1 cm/s, although other spray coating methods or batch coating or continuous flow coating methods and speeds can be utilized.

Step (1) includes stripping the coating and cladding of a commercial fiber 1102 using an aluminum razor and soaking the fiber in acetone (99.5%) at room temperature for 25 min to dissolve any remaining cladding. Note that this step would not typically be performed during commercial fabrication. The optical fiber was cut with a ceramic blade into 8.5 cm segments for the test fibers and 3.5 cm segments for the reference fibers. The clean fibers 1104 were individually attached to 3.0 cm ferrule connectors (SMO5SMA, Thorlabs) using 1/16″ and 3/32″ heat shrink tubes (Gardner Bender, New Berlin, Wis., HST-25), leaving 5.0 or 0 cm of the exposed optical fiber after polishing. The ferrule connector is a snug hollow tube with a threaded body that allows the optical fiber to be screwed into the polishing and optical setup. The fiber was mounted on a fiber support (D50SMA, Thorlabs) and polished using the optical polishing paper (LF30P, LF5P, and LF03P) to obtain a smooth surface on each circular face.

Step (2) includes dip-coating scattering centers 1106 onto the cleaned fiber core 1104. Aminated silica spheres (d=50, 100, 200, 400, and 500 nm) suspended in ethanol (99.99%) at room temperature (nanoComposix, San Diego, Calif., 10 mg/148 mL, SIANd-25M) were selected for two primary reasons. First, silica has low absorption in the UV range. Second, positively charged aminated spheres enable electrostatic attachment to the negatively charged glass core. Particles, such as amine-functionalized SiO₂, that have a positive surface charge below pH ˜7.5 allows excellent attachment of the positively charged particles to the glass fiber core, which has a negative surface charge above pH ˜2.5. Thus, a solution pH between about 3 and about 7 promotes attachment of the negative to positive surfaces. Each particle size was separately dip-coated onto different stripped optical fibers. Dip-coating involved submerging the fiber with tweezers into the aminated silica spheres suspension for 60 s and then allowing the fiber to air dry for 5 min. Dip-coating was repeated up to 7 times to deposit different masses of the silica scattering centers on the fiber. Gravimetric measurements (Sartorius M2P, Wood Dale, Ill., tolerance=0.01 mg) of noncoated and coated fibers determined the mass coverage (μg/cm²) of spheres on the fiber.

Step (3) includes submerging the coated fiber 1108 in variable aqueous ionic strength solutions 1110 (0-1 M) of sodium sulfate (Na₂SO₄) (Sigma-Aldrich, St. Louis, Mo., 239313) for 10 s at room temperature to increase the contact between the silica particles and the optical fiber core. The fiber was allowed to air dry for 5 min. In some cases, an ionic strength of the solution is at least about 0.1 M, at least about 0.2 M, at least about 0.3 M, or at least about 0.4 M.

Step (4) includes dip-coating the coated fiber 1108 with a polymer 1112 at a rate of 1 cm/s. Three polymers were used: (i) fluoropolymer (CYTOP), (ii) poly(methyl-methacrylate) (PMMA), and (iii) a common optical fiber polymer (DESOLITE). CYTOP was purchased as a polymer from BELLEX International Corp, Wilmington, Del. (CTX 109AE, RI: 1.34), dissolved in a nondisclosed perfluoro-compound at 9 wt %. PMMA powder (81489, RI: 1.48) was purchased from Sigma-Aldrich and dissolved in toluene (Sigma-Aldrich, 244511) at 9 wt %, 80° C., and 500 rpm. DESOLITE (DF-0016, RI: 1.370) was obtained from Desotech Inc., Elgin, Ill., as a liquid monomer and polymerized under UV 365 nm after dip-coating. A solid analysis of the optical fiber surface after each step was obtained by scanning electron microscopy with elemental mapping (SEM/EDX) (Philips XL30-EDAX) using gold and palladium sputtering.

Modifications of the above steps can be optimized to have uniform particle coating or tapered particle surface densities. Whereas treating the entire length of the fiber with the same ionic strength solution in Step (2) yields a consistent separation distance between the core surface and nanoparticle, tapering of ionic strength treatments in Step (2) (e.g., submerged into a 0.02 M, 0.05 M, 0.10 M and 0.15 M sodium sulfate solution at 6 cm, 12 cm, 18 cm 24 cm of fiber length (X) from the terminal fiber end that is connected to the light source, respectively) achieves variable separation distances (<10 to 100 nm) values along the length of the SEOF. Larger separation distance values near the light source (x=0) allow less light to be side-emitted at least because of lower interaction with the evanescent wave, whereas smaller separation distance values moving along the axial length of the SEOF typically have a larger percentage of the evanescent wave energy interacting the NP on the SEOF surface and side-emit more light.

Fabrication of LED-Optical Fiber Device. All optical fiber mounting parts were purchased from Thorlabs. A 30 mm cage system secured by four 8″ stainless steel rods (ER8) was used to secure and align all optical components. The 12 mW 265 nm UV-C LED (Boston Electronics, Brookline, Mass., VPC131) had a measured peak of 267 nm with a spectral width of 30 nm. The LED was secured by a cylindrical lens mount (CYCP), followed by three 1″ calcium fluoride uncoated plano-convex lens (LA5370), positioned by kinematic plates (KCl-T). The lenses maximized the light coupling into the optical fiber by capturing, coning, and focusing the light onto the optical fiber terminal end. Finally, the polished optical fiber 1114 was secured by the fiber adapter.

### Light Measurements

Localized Scattering Flux. Localized UV-C emission from the optical fibers was assessed by scattering flux measurements using a spectrophotoradiometer (Avantes, Louisville, Colo., AvaSpec-2048L, calibration: 200-1100 nm). The sensor tip of the spectrophotoradiometer (5 mm²) was placed normal and flush to the center of the optical fiber (2.5 cm from the ferrule connector). Only photons that are side-emitted are captured by the sensor, and the flux was obtained by integrating the output spectrum.

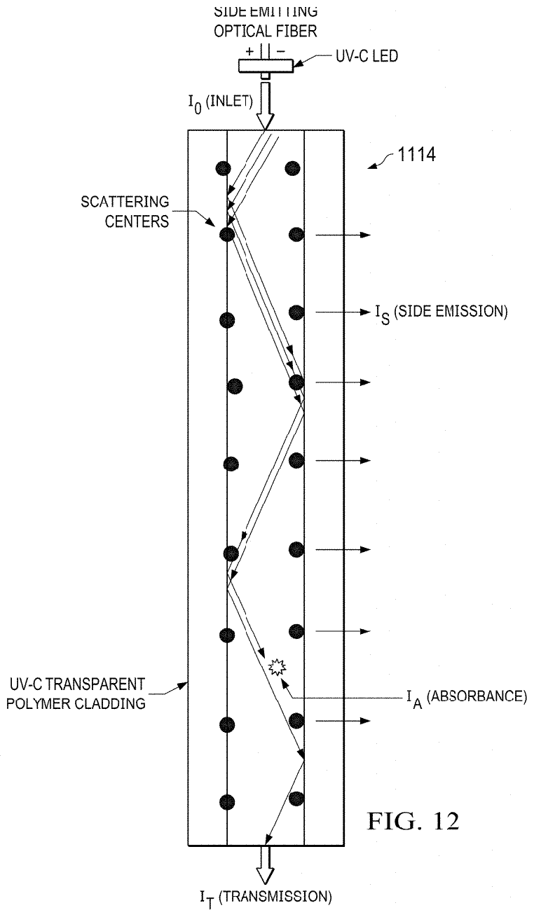

Total Scattering. Photons that are coupled into the optical fiber (I₀) can be transmitted (Iₜ) through the optical fiber core by internal reflection, absorbed (Iₐ) by the core or cladding materials or side-emitted (Iₛ) by scattering of the optical fiber 1114, as depicted in FIG. 12 and expressed in Eq. (1). Increasing the fraction of photons side-emitted from the optical fiber is beneficial for use in disinfection and is described as the scattering ratio (Iₛ/I₀). Both I₀ (photons/s) and J (photons/s) were quantified by potassium ferrioxalate actinometry experiments in the dark. All substances used in the actinometry experiments were purchased from Sigma-Aldrich.

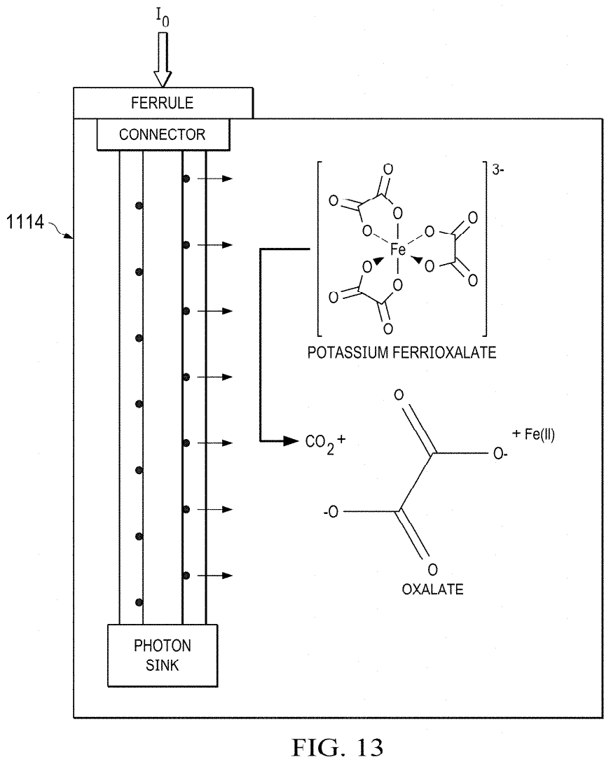

FIG. 13 depicts a potassium ferrioxalate actinometry experimental set up used to obtain total scattering from the optical fiber 1114. FIG. 13 depicts ferrule connector 1300 and casing 1302 encompassing the distal submerged terminal end of the optical fiber 1114 and serving as a photon sink for any light transmitted through the fiber. Reference (I₀) and test (Iₛ) optical fibers coupled to the UV-LED 265 were submerged in the 200 mL of the actinometry solution.

Chemical Actinometry. The light side-emitted from the fiber photoreduces potassium ferrioxalate, releasing Fe(II). To prevent the Fe(II) from reoxidizing to Fe(III), the solution was continuously purged with nitrogen gas. The experiment was conducted at room temperature (˜22° C.). A 2 mL sample was obtained from the solution at 0, 15, and 30 min, individually mixed with 1,10-phenanthroline, and left standing for 30 min. The concentration of the Fe(II)-phenanthroline red-colored complex was measured by the change in absorption at 510 nm using a UV-vis spectrometer (HACH DR5000, Loveland, Colo.). A control sample assured that no external light reached the actinometry solution. Finally, the quantum yield of the photoreduction of potassium ferrioxalate (Φ_{260nm<λ<300nm}=1.25) was used to calculate the photons side-emitted by the test optical fiber (Iₛ), transmitted by the test optical fiber (Iₜ), or transmitted by the reference optical fiber (I₀).

### Microbial Inactivation

Culture Preparation. The pure Escherichia coli culture was originally obtained from the American Type Culture Collection (ATCC 25922, Manassas, Va.). An inoculum from a frozen glycerol E. coli stock (kept at −80° C.) was streaked onto a fresh tryptic soy agar (TSA) plate and incubated overnight at 37±1° C. A single colony from the plate was recovered and inoculated into 5 mL of tryptic soy broth (TSB) and incubated overnight at 37±1° C. to start a liquid culture. The overnight TSB culture was diluted to a desired concentration and used for experiments. The absorbance spectra of the E. coli solution peaked at 268 nm, which coincides with the output LED spectra.

UV Exposure of E. coli Culture. The overnight culture of E. coli was diluted to approximately 5×10⁶ colony-forming unit (CFU) per mL using 10-fold dilution with the phosphate-buffered saline (PBS). The 0.5×PBS consisted of NaCl (0.0684 molarity), KCl (0.00134 molarity), Na₂HPO₄ (0.005 molarity), KH₂PO₄ (0.0009 molarity) with the final pH 7.4. A 5 mL aliquot of the diluted culture was transferred to a round-bottom polypropylene tube (12×74 mm²) covered with aluminum foil. The 5 cm UV-C SEOF was completely submerged in the center of the tube resulting in the 5.5 mm absorbance pathlength. E. coli culture was exposed to UV-C for 15, 30, and 60 min to illustrate a linear trend in inactivation over an hour of exposure. A control sample with no UV-C exposure was analyzed for each time to account for any photoreactivation mechanism from the treatment or enumeration environment. After each exposure time, duplicate samples were analyzed to quantify viable E. coli using the standard pour plate method. E. coli colonies were counted using Reichert Darkfield Quebec Colony Counter for plates containing 30-300 CFU/mL. Triplicates were obtained using different optical fibers.

To account for differences in UV-C transmittance between the E. coli and potassium ferrioxalate solutions, a correction factor (CF) was applied to the measured ferrioxalate actinometry UV-C dose. Using a UV-vis spectrometer with a 5.5 mm pathlength, the transmittance of 265 nm light (UVT265) for the E. coli solution was 75.5%, compared against only 2% for the potassium ferrioxalate solution. A CF value of 0.25 was calculated by dividing the fraction of light absorbed (UVA=1-UVT265) for the E. coli solution (24.5%) by that of the ferrioxalate actinometry solution (98%). The corrected UV-C dose reported for E. coli experiments is the dose (mJ/cm²) determined by ferrioxalate actinometry multiplied by CF.

Pour Plate Method. TSA medium was prepared according to the manufacturer's instructions. Briefly, 40 g of TSA powder (Sigma-Aldrich, 22091) was dissolved in 1 L of deionized water while heating (at maximum heat) under constant mixing using a stir bar. After complete TSA dissolution, the medium was autoclaved at 121° C. for 15 min. The autoclaved medium was kept in a water bath set at 48±2° C. The E. coli cultures exposed to UV-C were serially diluted (10-fold dilution) using 1×PBS. All dilutions were analyzed in duplicate. Briefly, 1 mL of each dilution was added to a sterile Petri dish, followed by the addition of 15 mL of liquified TSA medium acclimatized at 48±2° C. The plates were quickly swirled to thoroughly mix the liquid TSA with the sample and then left undisturbed in a biosafety hood for 30 min or until the medium was completely solidified. The plates were incubated at 37±1° C. for 48 h, and data was recorded as colony forming units (cfu) per mL. Disinfection efficacy of the coated, side-scattering fiber optic probe for each contact time was calculated using the mean log reduction (LR) for E. coli.

Inactivation of bacteria on nutrient rich surfaces in air. Bertani (LB) broth media was prepared according to manufacturer instructions. Briefly, 25 g of LB broth powder (22091, Sigma-Aldrich, St. Louis, Mo. USA) was added to 1 L of ultrapure water and autoclaved for 15 minutes at 121° C. P. aeruginosa (ATCC 15692) and E. coli (ATCC 25922) were grown in the media on a shaker plate at 140 rpm in an Isotemp incubator (MaxQ 400, Fisher Scientific, Hampton, N.H. USA) at 37° C. for 12 hours. The culture was diluted in LB broth (1:10) and grown in the same conditions until it reached an absorption of 1 cm⁻¹ as measured by a spectrophotometer (Odyssey DR/2500, HACH, Loveland, Colo. USA). The culture was washed 3 times and resuspended in the wash solution to eliminate UV absorption by the media. The wash solution was prepared by diluting 0.9 wt % of sodium chloride (S7653, Sigma-Aldrich) in nano purified water and autoclaved for 15 minutes at 121° C. The process was repeated for each experiment.

The zone of inhibition of the UV-C side emitting optical fiber (SEOF) was measured by placing the fiber on an agar plate spread with a P. aeruginosa culture. The LB broth agar plates were prepared by dissolving 10 g of tryptic soy agar (2291, Sigma-Aldrich) in 1 L of LB broth media. The solution was autoclaved, cooled, and poured in 10 cm² gridded polystyrene square petri dishes (741470, Carolina, Burlington, N.C. USA). Once the media solidified, 50 μL, of the P. aeruginosa or E. coli solution was spread across the agar to form a lawn as previously described in inhibition zone studies. The UV-C SEOF was immediately positioned directly above the agar through two small holes on the side of the plate. The plate remained closed to avoid unwanted contamination. The plates were exposed to UV-C for 0, 30, 60, 120, 249, and 480 min in random order. Triplicates data were obtained using different optical fibers and plates. Student t-test was used to measure the statistical significance of the results where key t-test assumptions were met.

Two controls were analyzed. In the first control no fiber was added to the plate to visualize a healthy lawn formation. In the second control, the fiber was placed on the plate without turning on the 265 nm LED. This assured that the material properties of the fiber did not have germicidal effects. Additionally, a bare optical fiber was analyzed for comparison. This fiber was stripped of the previous coating and coated with the UV transparent polymer without SiO₂ modification. After UV-C exposure, the plates were incubated at 37±1° C. for 24 h. The distance between the optical fiber and the P. aeruginosa lawns on either side of the optical fiber was measured and recorded as the zone of inhibition. The measurement was taken at 0, 2, 4, 6, and 8 cm along the optical fiber length to understand how light attenuation affects zone of inhibition.

### Material Characterization

A UV-vis spectrophotometer (PerkinElmer, LAMBDA 950 UV/VIS, Waltham, Mass.) equipped with a Spectralon surface (Labsphere, North Sutton, N.H.) integrating sphere was used to measure the absorption of the materials used in this study. The absorbance (A) of the aminated silica spheres was obtained by diffuse reflective spectroscopy (DRS) and corrected using the Kubelka-Munk equation. A 1″×1″ quartz substrate (Ted Pella Inc., 26012) was prepared by dripping 300 μL of 99.99% ethanol-suspended aminated silica on the substrate and letting it dry at room temperature for 30 min. This resulted in 4.65 (±6%) μg/mm² loading. The cycle was repeated 10 times, until the sample was visually opaque. The sample was then placed in the back of the integrating sphere so that any scattering/reflection was measured as T. For the polymer absorption measurements, quartz substrates were prepared by dip-coating at 1 cm/s (same rate as the fiber preparation). The sample was placed in front of the integrating sphere.

Transmission measurements were obtained at 265 nm and reported as absorbance, where A=1−T, assuming insignificant reflection/scattering. There was no noticeable difference in transmission through the spectral output of the LED. Thickness was measured by a stylus profilometer (Bruker XT) and was 5-20 μm. The absorption was adjusted to 5 μm per the Beer-Lambert law.

### Results and Discussion

Two factors that can influence scattering flux of UV-C through the optical fiber length were explored: (i) extinction (absorption and scattering) over the cross-sectional area of the material being used as a scattering center and (ii) loading of scattering centers, which influence how many particles interact with photons. The interactions between photon and scattering centers are possible via two means: (i) light refracting from the quartz fiber waveguide at the contact point between the scattering center and optical fiber core or (ii) the interaction of particles with the evanescent wave. Evanescent waves are the electromagnetic disturbance formed by total internal reflection at the interface of the transmitted medium. The wave amplitude decays exponentially with the distance from the interface. Designing the scattering center materials and loading led to maximum utilization (i.e., side emission) of LED light entering the optical fiber, which enabled microbial disinfection. Each of these factors is described below.

Selecting Low UV-C Absorbing Scattering Centers. A particle's extinction coefficient is a measure of the amount of light removed from a beam that comes in contact with it normalized by the geometric cross-sectional area of the particle that is normal to the beam. Here, the light removed from the beam by the particle is either absorbed (absorption cross-sectional area) or scattered (scattering cross-sectional area). For side emission of light from optical fibers, particles with low absorption and high scattering of UV-C can be selected. The theoretical scattering and absorption cross-sectional area of gold, silver, and silica were predicted based on Oldenburg's Mie theory. Silica spheres were selected as the scattering centers for the UV side-emitting optical fibers due to significantly higher scattering cross-sectional area than absorption cross-sectional area, regardless of the particle size.

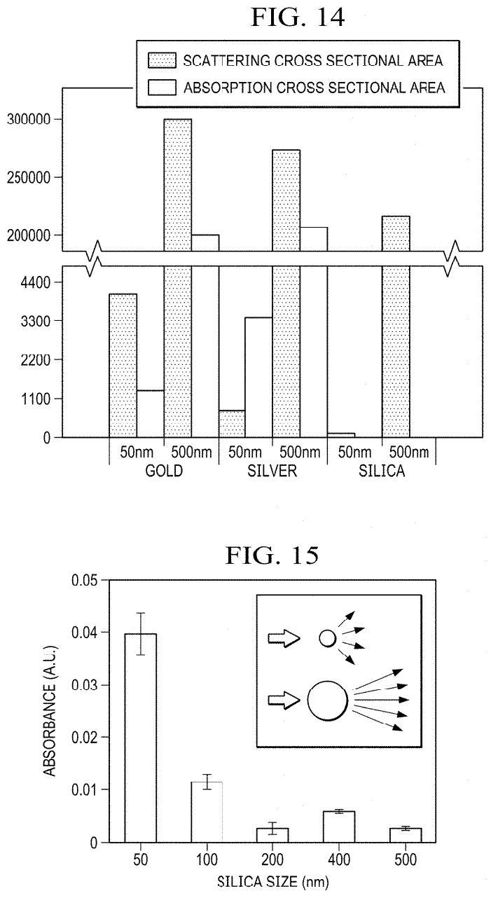

FIG. 14 shows theoretical scattering and absorption cross-sectional area of gold, silver, and silica predicted based on Oldenburg's Mie theory calculator. For each size particle, the scattering cross-sectional area is depicted by the bar on the left, and the absorption cross-sectional area is depicted by the bar on the right. The extinction coefficient is the amount of light a particle removes from a beam normalized by the geometric cross sectional area of the particle that is normal to the beam. Here, the light is either absorbed (absorption cross sectional area) or scattered (scattering cross sectional area). The silica spheres illustrated negligible absorption for both 50 nm and 500 nm diameters (no bars visible), negligible scattering for the 50 nm spheres, and significant scattering for the 500 nm spheres.

FIG. 15 shows the absorbance at 265 nm of silica spheres between 50 and 500 nm. The absorbance of the aminated silica spheres deposited on a quartz substrate was obtained by diffuse reflective spectroscopy (DRS) and corrected using the Kubelka-Munk equation. During DRS, light cannot be transmitted and, is, therefore, either absorbed or detected by scattering/reflection. Smaller particles (50 nm diameter) absorb more and scatter less 265 nm light than larger silica spheres (>200 nm diameter), which absorb very little light and instead scatter it (FIG. 15 inset). This result is explained by the Mie theory, where I_{A}/I_{S} increases with the decreasing diameter because the particle scattering cross-sectional area decreases in proportion to its volume for d<2. These measurements suggest that larger diameter silica spheres should lead to the preferred outcome of scattering, rather than absorbing, 265 nm light if placed on the glass fiber core.

Maximizing UV-C Scattering from the Optical Fiber by Varying Silica Sphere Diameter and Loading. The diameter of silica used as the scattering center affected the scattering the cross-sectional area as well as the amount of interactions that result in light scattering. Steps (1) and (2) of the fabrication process were followed with each diameter silica sphere. Smaller silica spheres have higher interfacial contact with the optical fiber core. Higher silica sphere loading can also contribute to increased scattering until it plateaus.

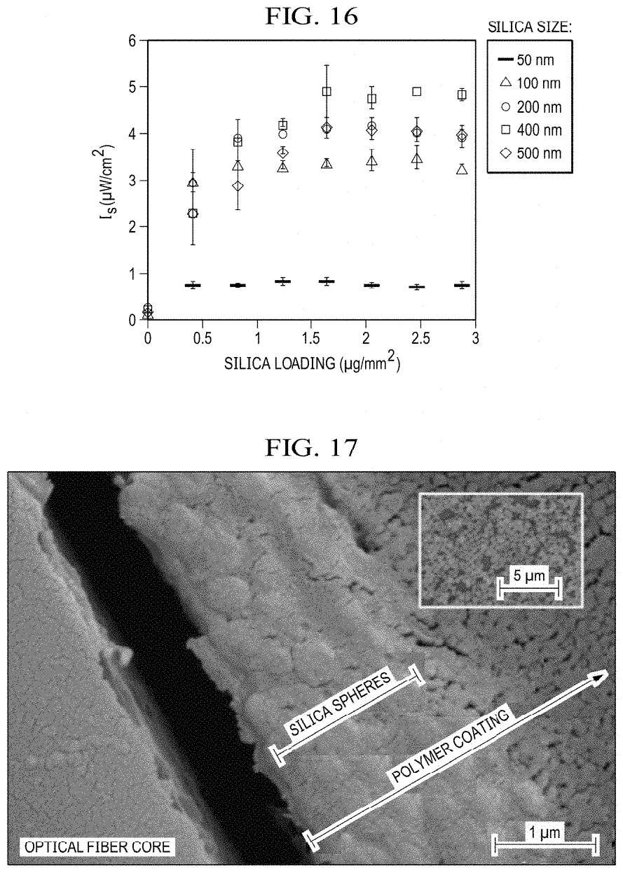

FIG. 16 shows the measured scattered light halfway down the optical fiber (2.5 cm) after coating the fiber with variable mass loadings (0-3 μg/mm²) of different diameter silica spheres (50, 100, 200, 400, and 500 nm). Particle loading was varied by the number of dip-coating cycles. Each dipping cycle resulted in (0.41 μg/mm²±6%) additional loading for all of the sizes. Electron microscopy images confirmed continuous layers of silica spheres on the surface of the optical fibers.

FIG. 17 shows a scanning electron microscope (SEM) image of optical fiber cross section after the four-step preparation process depicted in FIG. 11. The image was obtained at 20.00 kV and 20,000× magnification. Illustrated are the optical fiber core (left) and the silica spheres coated by CYTOP (right). The space between the optical fiber core and silica spheres is an artifact of the SEM and was formed during microscopy. The inset illustrates a surface SEM of the arrangement of the silica spheres on top of the optical fiber core without the CYTOP, illustrating that the silica spheres do not form a monolayer. For the 400 and 500 nm silica, higher loading results in higher scattering. As expected, a plateau in scattering was reached for every size, such that increasing the loading of silica spheres onto the fiber does not result in increased scattering.

Particles diameters of 200 nm or greater achieved similar scattering according to the student t-test with 95% confidence level (4.4 μW/cm²; p>0.10), with slightly more scattering than 100 nm diameter particles (3.4 μW/cm²; p=0.023) and more than 5-fold higher scattering than 50 nm spheres. Spheres having an average diameter of at least 200 nm provide effective UV-C side emission from optical fibers.

Optional High Ionic Strength Treatment Increases Side-Emission of UV-C Light. The benefits of enhanced side emission of light associated with an optional post-treatment step using a high ionic strength solution (Step (3)) were observed. In developing the method, Step (2) was performed at different pH levels to investigate electrostatic interactions between the aminated silica spheres and the glass fiber core. However, the scattering significantly increased at a lower pH. Similar results occurred using sulfuric or hydrochloric acid. Subsequent experiments using high molarity sodium sulfate solutions demonstrated that the improved scattering was due to higher ionic strength, and results depended less on pH. As used herein, “ionic strength” a measure of the strength of the electric field in a solution, equal to the sum of the molarities of each type of ion present multiplied by the square of their charges.

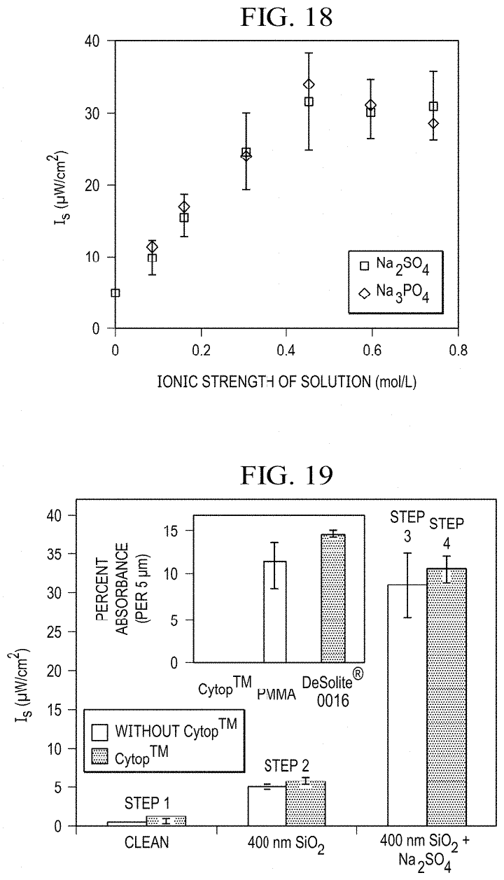

FIG. 18 shows representative data, with increasing scattering halfway down the optical fiber for different ionic strength solutions using 400 nm silica spheres. For treatment with ionic strength >0.45 M (0.2 M Na₂SO₄), the scattered flux reached greater than 6-fold higher than without Step (3). To verify that the cause of increased scattering was ionic strength, the same experiment was conducted with Na₂PO₄, normalizing for ionic strength. The scattering increase results are within the standard deviation for Na₂SO₄. Thus, subsequent fibers were prepared using 0.2 M Na₂SO₄ in Step (3).

Three causes were speculated for the increase in scattering after high ionic strength treatment. First, salt precipitate on the surface of the optical fiber could create additional areas of uniformity. However, neither sodium nor sulfur was present on the optical fiber surface when examined using scanning electron microscopy (SEM) with elemental mapping. Second, high ionic strength could cause the particles to rearrange and form a more uniform monolayer. However, SEM images of the fiber surface did not illustrate a significant change in arrangement pre- and post-high ionic strength treatment. Third, high ionic strength treatment likely compressed the electric double layer around the aminated spheres, packing them closer to the surface. The third explanation may explain the observed enhanced side-emitting light.

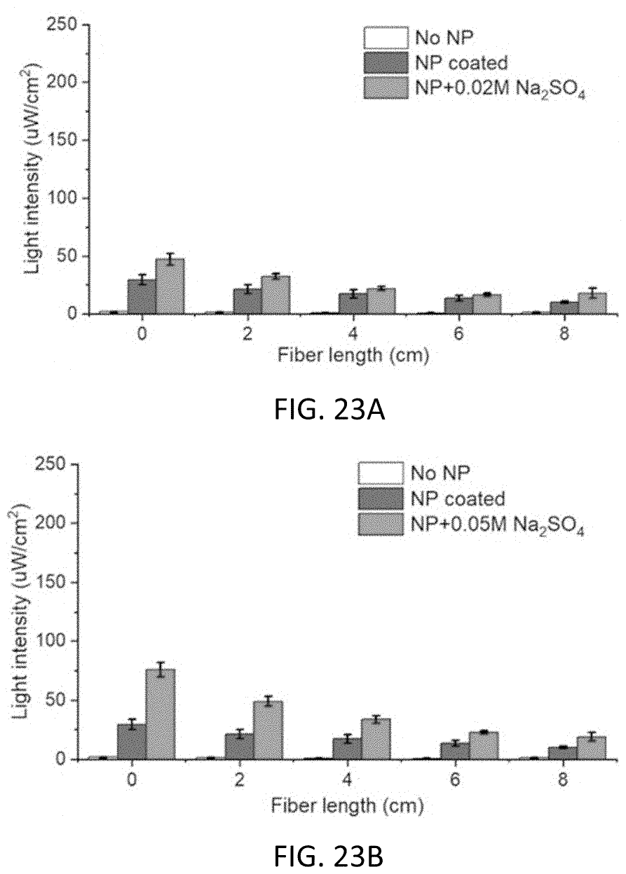

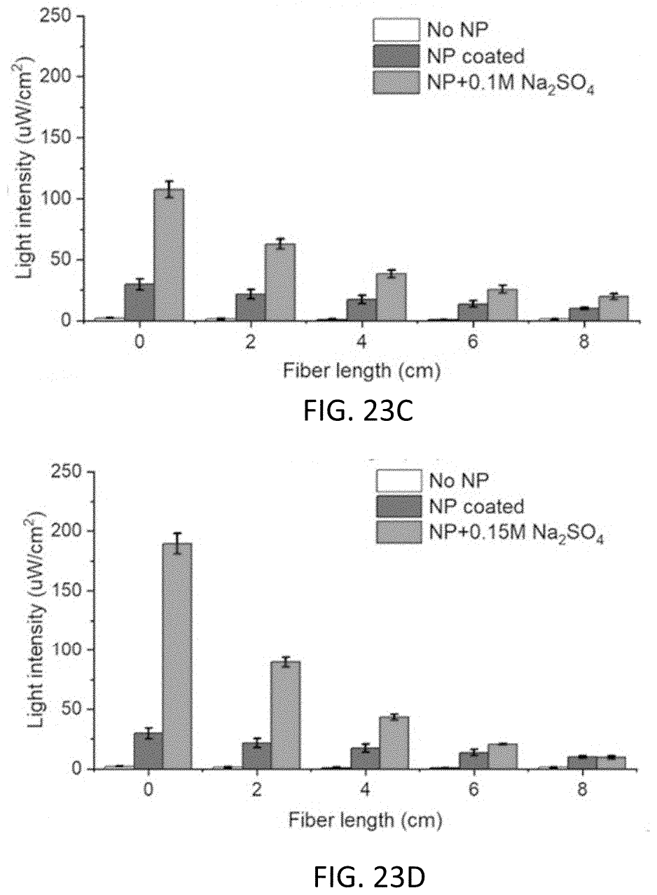

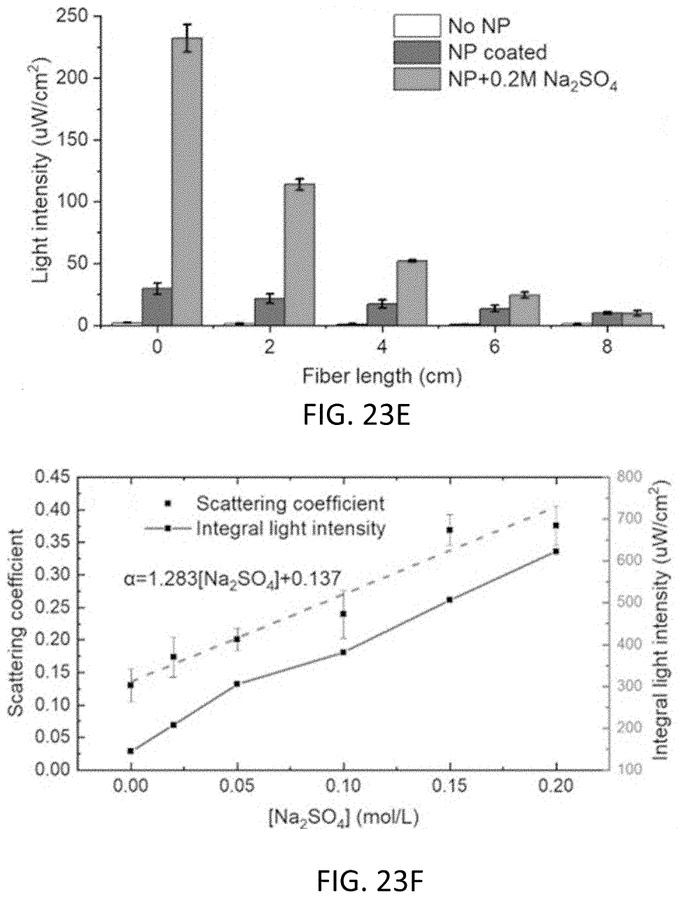

FIGS. 23A-23F show data from SEOFs created using 200 nm aminated silica sphere nanoparticles (suspended in 99.99% ethanol, 10 mg/mL, nanoComposix, San Diego, Calif.) and post-treated using variable ionic strength treatments, before being coated with UV-C transparent polymer. FIGS. 23A-23E show UV-C light irradiance measured at different distances along an optical fiber with 0.02M Na₂SO₄, 0.05M Na₂SO₄, 0.10M Na₂SO₄, 0.15M Na₂SO₄, and 0.20M Na₂SO₄, respectively. FIG. 23F shows linear fitting of scattering coefficient (left) and integrated light intensity (right) with the concentration of Na₂SO₄ used on the SEOF. The standard deviations for triplicate independent optical fibers are illustrated as error bars. Light intensities were greatest near the LED source (i.e., fiber length (cm) closer to zero) and then exponentially decreased with longer distances from the LED source. In all cases, inclusion of the ionic strength treatment lead to 2× to nearly 10× higher side-emitted light intensities at any length along the fiber. There was ˜60% decrease of side-emitted light intensity from the proximal (L=0 cm) to distal (L=8 cm) end without the ionic strength treatment and larger drops in side-emitted light intensity of 66%, 75%, 81%, 94% and 96% when 0.02M, 0.05M, 0.1M, 0.15M and 0.2M sodium sulfate were applied, respectively. As more light is side-scattered from the SEOF near the LED source (i.e., with higher ionic strength treatment) then there is less light available to travel down the length of the fiber. Scattering coefficient (α) values were calculated

$$( a = \frac{- ln \frac{{I}_{s} ( {x}_{2} )}{{I}_{s} ( {x}_{1} )}}{• x} )$$

for each experiment. FIG. 23F shows that α values were linearly correlated with the ionic strength utilized during the coating process: α=1.28*[Na₂SO₄, M]+0.137.

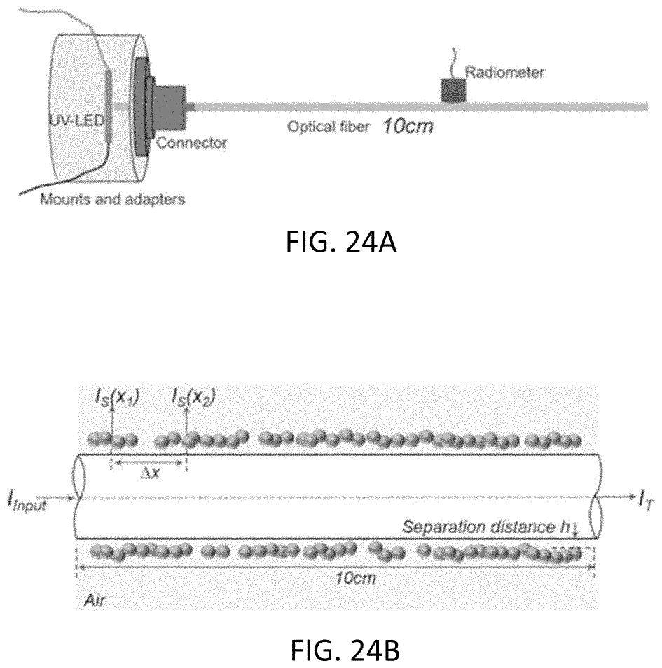

FIGS. 24A and 24B show schematically how ionic strength impacts separation distances between the aminated silica nanoparticles and an optical fiber glass surface, thus increasing the light scattering through greater interaction with the evanescent wave energy.

A MATLAB based predictive model was used to simulate the effect of separation distance (0 nm to 100 nm) on the light scattering along optical fiber. Model conditions mirrored experimental conditions (e.g., 200 nm particle size, 0.45 A current with a 265 nm UV-C light with the emission profile provided by the manufacturer, 0.39NA optical fiber, CyTop polymer coating). The mathematic predictive model was developed and run in MATLAB version R2019b to generate light profiles (μW/cm²) along the optical fiber length. Fiber parameters (length, diameter, material), nanoparticle parameters (size, density, placement) and system parameters (angle resolution, iterations, optional polymer coating, distance between radiometer and fiber) were input into the main file. Computational raytracing was used to calculate theoretical side-emission profiles. The light profile from the LED was divided into a series of small sections, and the ray was tracked down the length of the fiber, with trajectory and intensity governed by the laws of the model. Three phenomena were involved in this model: linear optics (Fresnel and Snell equations), which can govern the trajectory and intensity of the light reflecting inside the optical fiber; evanescent wave, which occurs when a ray hits the edge of the fiber where a nanoparticle exists; and Mie scattering, which occurs when any amount of light interacts with a nanoparticle. The Mie profiles for the particles were generated using MiePlot v4.6.14.

UV-C light irradiance was simulated at different distances along the optical fiber with separation distances (h) ranging from 1 nm to 100 nm between the NP and SEOF surface. The model is consistent with the experimentally observed exponential decrease in side emitted light and magnitudes of light intensity (see, e.g., FIGS. 23A-23F).

Thus, controlling the separation distance between particles and the core surface plays a role in controlling the amount of side-emitted 265 nm light from the fiber, along the length of the fiber. Ionic strength treatment compresses the electric double layer on the surfaces, allowing the particles to be located closer to the core surface (i.e., shorter separation distances). Ionic strength treatment is one way to achieve, and control, the separation distances. Other methods can include depositing thin layers (<100 nm thick) of UV-C transparent polymer containing particles on the optical fiber surface.

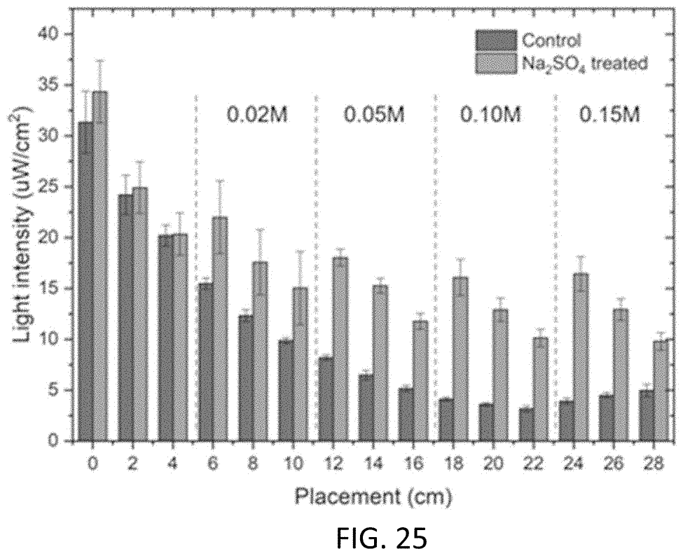

FIG. 25 demonstrates how tapering the ionic strength can change the amount of light that is side-emitted as a function of distance from the distal end. The SEOF was treated using a tapering of ionic strengths (submerged into a 0.02 M, 0.05 M, 0.10 M and 0.15 M sodium sulfate solution at 6 cm, 12 cm, 18 cm, and 24 cm, respectively) to achieve variable h values along the length of the SEOF. Higher h values near the LED (x=0) allows less light to be side-emitted because of less interaction with the evanescent wave, whereas lower h values moving along the axial length of the SEOF allow a larger percentage of the evanescent wave energy interacting the NP on the SEOF surface and side-emit more light. This demonstrates the tunability of side-emitted light by modulating the separation distance between particles and the core fiber surface.

Polymer Coating Material Effect on Scattering. A technological barrier for UV-C side-emitting optical fibers is that even if the light is scattered away from the core of the optical fiber, it gets absorbed by the polymer cladding. Step (4) in preparing the UV-C emitting optical fiber involves selecting and applying a UV-transparent (UV-C transparent) polymer cladding. The cladding serves dual purposes of reflecting light within the fiber (total internal reflection) and physically protecting the fiber. High optical transmittance of the polymeric coating is essential in side-emitting optical fibers because the light must go through the polymer before it can inactivate microorganisms. Three different polymers were considered: DESOLITE 0016, PMMA, and CYTOP. CYTOP is a transparent fluorosis with a low refractive index that has >95% transmittance (200 μm thickness) for UV 265 nm wavelength. Additionally, CYTOP has a lower index of refraction than the silica core. The polymers were deposited on a quartz substrate, and absorbance of UV 265 nm was measured using diffuse reflective spectroscopy (FIG. 19 inset). Thicknesses of 5-20 μm were measured using a stylus profilometer, and absorption was adjusted to 5 μm per the Beer-Lambert law. PMMA and DESOLITE absorbed 11.1 and 14.8% of UV 265 nm, respectively. A complete transmission was observed by CYTOP, indicating the null absorption of 265 nm light. Therefore, CYTOP was selected as the polymer coating for Step (4) in the UV-C side-emitting optical fiber fabrication.

To better understand its interaction with silica spheres before and after high ionic strength treatment, the optical fiber was coated with CYTOP after each preparation step. The localized scattering flux was measured by a spectrophotoradiometer halfway through the fiber (2.5 cm from the ferrule connector). FIG. 19 depicts the scattering flux of each preparation step (clean core, 400 nm SiO₂, 400 nm SiO₂+Na₂SO₄) before and after applying CYTOP. There is a slight increase in scattering after applying CYTOP to the clean optical fiber (1.1 μW/cm²±4%). This is likely due to impurities in the polymer as this was not done in a clean room. There was no statistically significant difference in scattering before and after the coating was applied to the 400 nm silica-coated fiber (p=0.31) or the Na₂SO₄-treated fiber (p=0.66). This supports the claim that CYTOP does not affect the side-emission of the optical fiber to be used in microbial inactivation.

FIG. 19 additionally compares the effect of each preparation step of the process depicted in FIG. 11 on the flux emission. The stripped fiber averaged a scattering flux of 0.2 μW/cm². Adding 400 nm silica spheres to the surface of the fiber core (step 2) improved scattering 37-fold to 8.0 μW/cm². Treating the fiber with a solution of high ionic strength increased the localized scattering flux an additional 3.6 times to 36.9 μW/cm². Ionic strength treatment without silica spheres resulted in no significant increase in scattering from the clean fiber (0.23 μW/cm²).

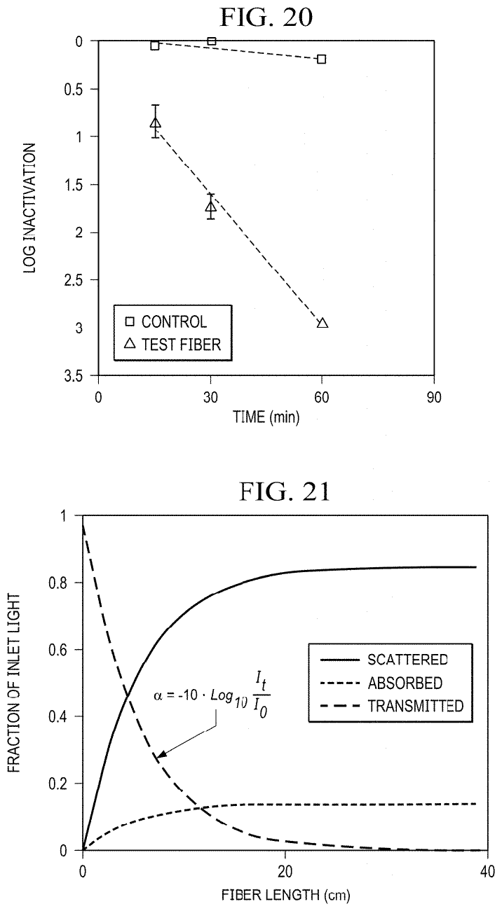

Microbial Inactivation in water by UV-C Side-Emitting Optical Fiber. FIG. 20 shows the log inactivation of E. coli by coupling a UV 265 nm wavelength LED to a side-emitting optical fiber. Two optical fibers were prepared for E. coli inactivation. The side-emitting optical fiber (solid triangles) included all 4 preparation steps, whereas the control (solid squares) included only steps 1 and 4 (the clean fiber coated with CYTOP) to assure that the CYTOP was not contributing to the inactivation of E. coli. The illuminated optical fiber was submersed in the 5 mL polypropylene tube filled with E. coli solution, as described in the methods. The total photons emitted by the side of the optical fiber were measured by potassium ferrioxalate actinometry and are defined as the delivery dose.

The test optical fiber achieved 2.9 log inactivation for a dose of 15 mJ/cm²; this equated to 1 h of operation using the low power LED. It is known that the experimental setup and exposure conditions can impact the UV doses required for the inactivation of different types of bacteria. UV-C doses of 8-6 mJ/cm² can result in 3 log₁₀ inactivation of washed E. coli (ATCC 29425) cultures. Here, the side-scattering optical fiber for the inactivation of the unwashed culture of E. coli, directly diluted in PBS. The unwashed culture of test bacteria is expected to contain greater residual organics from nutrient media, which might be responsible for the relatively high UV dose reported here for the inactivation of E. coli. Additionally, the reactivation of bacteria after low UV-C dose exposure is well documented and can lead to a higher required dose for similar inactivation potential. Here, microorganisms can recover activity through repairing pyrimidine dimers in the DNA after damage by low UV-C doses.

The control optical fiber without scattering centers achieved 0.2 log inactivation for a delivery dose of 4 mJ/cm² over the same 1 h exposure. This illustrates that the CYTOP coating did not have major germicidal effect for E. coli (i.e., it did not damage the cells' DNA and that the side-emitting UV-C radiation caused the inactivation. The silica sphere-modified optical fiber delivered >3.5 times the UV-C dose and obtained 16-fold higher inactivation than when only CYTOP was applied. The results demonstrate that UV-C side-emitting optical fiber can be used to inactivate E. coli. In this work, 25% of the radiation applied to the optical fiber was emitted through the side of the optical fiber. Higher output UV-LED or better coupling of light into the optical fiber would increase the intensity of UV-C side emission.

Microbial Inactivation on a nutrient-rich surface in air by UV-C Side-Emitting Optical Fiber. Light distribution along the optical fiber length was observed. Images were captured under dark conditions with a paper towel placed below the optical fiber. The paper towel fluoresces blue light upon ultraviolet irradiance, allowing for visualization of the light distribution throughout the optical fiber. Bright spots seen towards the top of the images were due at least in part to (i) light leaving the distal end (opposite end as the light source) of the optical fiber and (ii) back reflection at the distal end of the fiber. When the light reaches the distal end, most of it exits the fiber. However, a portion of light also reflects towards the optical fiber creating a second “input” and higher photo density at the distal end.

The nanoparticles on the SEOF interacted with the evanescent wave and resulted in side-emission through scattering, creating a visible side emission “glow.” This glowing germicidal light enabled microbial inactivation along the length of the optical fiber. The observable inactivation along the length of the bare optical fiber is due to natural light scattering that results at least in part from surface impurities since these fibers were not prepared in a clean room. Additionally, the zone of inhibition increases slightly towards the distal end of the fiber.

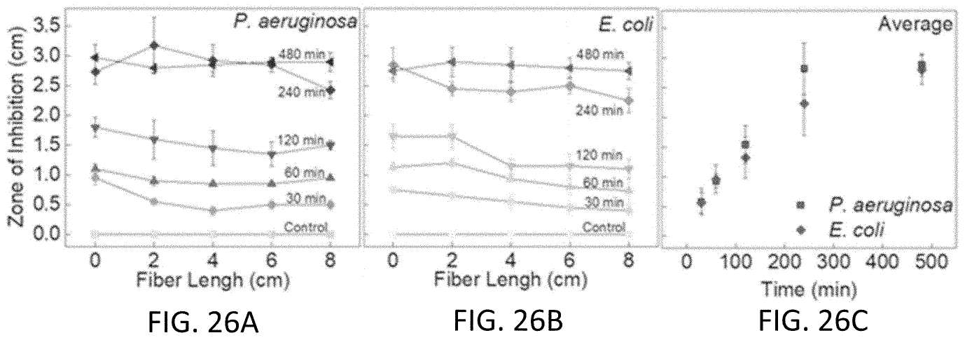

FIGS. 26A and 26B show zones of inhibition of P. aeruginosa and E. coli, respectively,) resulting from UV-C SEOF exposure on an agar plate lawn after 0, 30, 60, 120, 240, and 480 minutes. At each exposure time, the zone of inhibition was measured at L=0, 2, 4, 6, and 8 cm from the proximal end of the LED source along the length of the fiber. A dark control was obtained by placing a modified optical fiber in the agar plate without turning on the UV source. No inhibition was observed by the dark control, indicating that the exterior materials of the optical fiber did not contribute to the germicidal effect of the UV-C SEOF.

FIGS. 26A and 26B show that the zone of inhibition was higher at the proximal end and lower towards the distal end for lower exposure times (t<120 min) with both P. aeruginosa and E. coli. This observation corresponds with the UV-C side emission profile, as light side emits from the optical fiber, the photon density decreases inside the fiber. This phenomenon is described by the Beer-Lambert law of attenuation through a waveguide. A lower photon density means less light can be emitted through the fiber's side. However, the path of side emitted photons is not directly normal to the optical fiber. At increasing distance from the optical fiber, the localized irradiance is a sum of the irradiance emitted from the entire length of the optical fiber.

FIG. 26C shows that linear increase in zone of inhibition with time reaches a maximum of ˜2.9 cm at around 240 minutes of irradiation. The UV dose at the edge of the lawn at 240 minutes is approximately 4.3 mJ/cm² (irradiance=0.3 μW/cm²). Between 240 and 480 minutes there is no statistically significant change in zone of inhibition for either organisms according to the Student t-test with 95% confidence level (p>0.05). The irradiance at 480 minutes is also 0.3 μW/cm² resulting in double the dose (8.6 mJ/cm²) for the same zone of inhibition. Additionally, single 12-hour (12.9 mJ/s) and 24-hour (25.8 mJ/s) exposure times resulted in <3.0 cm zone of inhibition. These results indicate that there is a maximum zone of inhibition (MZI) that is not solely dependent on dose.

At the edge of the inhibition zone, the localized irradiance is insufficient to either (i) damage the DNA and protein of the organism or (ii) surpass the rate of DNA and protein reconstruction. UV-C radiation at 265 nm is categorized as germicidal because it inhibits pathogens (i.e., bacteria, virus, protozoa) from replicating and infecting a host. Absorption of UV light by nucleic acids results in crosslinking between thymine and cytosine. These mutations disable hydrogen bonds to the purine base of the opposite strand, therefore inhibiting replication. This process reverts DNA back into its undamaged form. At the low localized irradiance beyond the MZI then DNA repair rates may exceed DNA damage rates, thus limiting net inactivation of the microorganism.

The MZI depends at least in part on (i) the sensitivity of the microorganism to UV light and (ii) the input power of the LED. By Student's t-test, there is no statistical difference in either MZI or zone of inhibition (p>0.05) at each irradiation time of P. aeruginosa and E. coli through the entire fiber length. This result is supported by similar UV sensitivity reported for these organisms. For 4-log (i.e., 99.99%) inactivation, doses range between 3.1 and 17 mJ/cm² for planktonic P. aeruginosa and between 3.0 and 20 mJ/cm² for planktonic E. coli (e.g., FIG. 20).

Scaling Fabrication of UV-C Side-Emitting Optical Fibers. A scalable method of modifying optical fibers to side-emit UV-C radiation has been described. This process can be adapted to large-scale fabrication. In the commercial-scale optical fiber production, the melted, thinned, and cooled glass core is pulled through a series of coating dies and drying ovens. For UV-C side-emitting optical fiber fabrication, the first die would contain a solution of aminated silica spheres. The fiber would then be rolled through a high ionic strength solution before entering the final die containing CYTOP.

The logarithmic decay (Beer-Lambert law) of light through the optical fiber shown in FIG. 21 that could cause uneven inactivation effectiveness along the fiber length can be mitigated in at least two ways. First, the silica sphere loading or side-emitting efficiency can be modulated by either varying the loading of 400 nm silica spheres along the length of the optical fiber or by varying the length of the optical fiber exposed to high ionic strength solutions. Second, light can be supplied from both ends (proximal and terminal sides) of the optical fiber using two LEDs. Coupling the optical fibers to a higher output LED will decrease the retention time needed for inactivation.

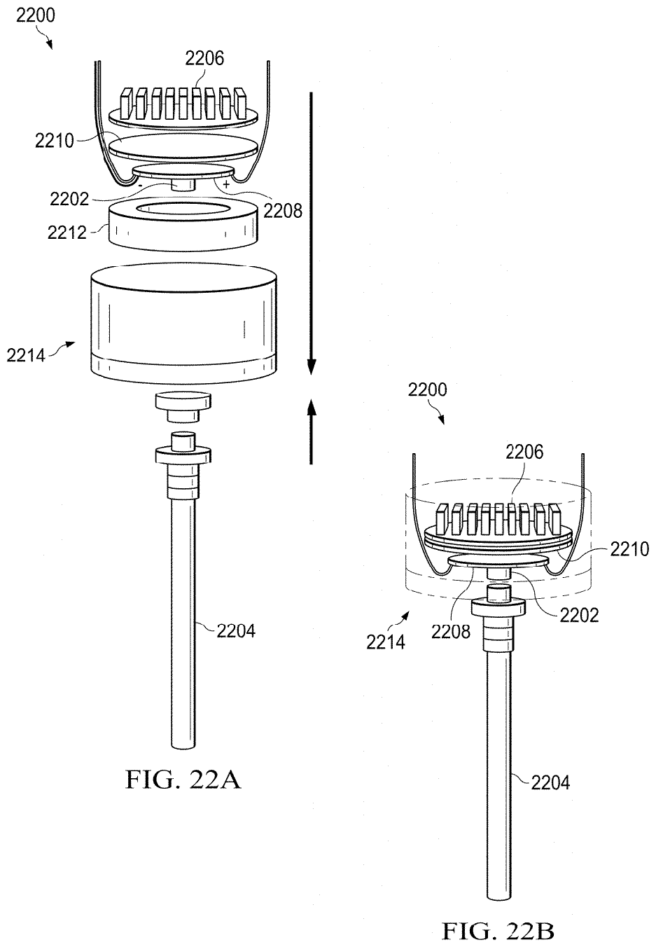

FIG. 22A is an exploded view of device 2200 including a UV-C LED 2202 coupled to a UV-C side-emitting optical fiber 2204. Device 2200 includes heat sink 2206 coupled to mounting board 2208. Metal plate 2210 is positioned between heat sink 2206 and mounting board 2208. In one example, heat sink 2206 is made of aluminum and metal plate 2210 is made of copper. O-ring 2212 is provided between optical fiber 2204 and mounters and adapters 2214. FIG. 22B is an assembled view of device 2200 depicted in FIG. 22A. As depicted in FIG. 22B, an end of optical fiber 2204 is nearly touching LED 2202 to optimize light coupling.

Only a few implementations are described and illustrated. Variations, enhancements and improvements of the described implementations and other implementations can be made based on what is described and illustrated in this document.

What is claimed is:

1. A coated optical fiber comprising:
a core, wherein the core is substantially UV-transparent;
particles optically coupled to the core; and
a polymer coating in contact with the particles, wherein the polymer coating is substantially UV-transparent.

2. The coated optical fiber of claim 1, wherein the particles comprise silica beads.

3. The coated optical fiber of claim 2, wherein the particles comprise aminated silica beads.

4. The coated optical fiber of claim 1, wherein an average diameter of the particles is in a range from about 50 nm to about 500 nm.

5. The coated optical fiber of claim 4, wherein the average diameter of the particles is in a range from about 200 nm to about 500 nm.

6. The coated optical fiber of claim 5, wherein the average diameter of the particles is in a range from about 200 nm to about 400 nm.

7. The coated optical fiber of claim 1, wherein UV light passing through the core is scattered by the particles through the polymer coating.

8. The coated optical fiber of claim 1, wherein a thickness of the polymer coating is between about 10 μm and about 100 μm.

9. The coated optical fiber of claim 1, wherein the particles comprise about 0.5 wt % to about 2 wt % of the polymer coating.

10. A disinfectant system comprising the coated optical fiber of claim 1.

11. An apparatus comprising a light source optically coupled to the coated optical fiber of claim 1.

12. The apparatus of claim 11, wherein the light source comprises a light-emitting diode (LED).

13. The apparatus of claim 12, wherein the light source comprises a UV-C LED.

14. The apparatus of claim 11, wherein the light source is thermally coupled to a heat sink.

15. A method of coating an optical fiber, the optical fiber comprising a core, and the method comprising:
optically coupling particles to a surface of the core to yield a functionalized core;
coating the functionalized core with a polymerizable material; and
polymerizing the polymerizerable material to yield a substantially UV-transparent polymer coating on the functionalized core.

16. The method of claim 15, wherein optically coupling the particles to the surface of the core comprises adhering the particles to the surface of the core.

17. The method of claim 15, wherein the particles comprise about 0.5 wt % to 5 wt % of the polymerizable material.

18. The method of claim 15, further comprising contacting the functionalized core with an aqueous solution having an ionic strength of at least about 0.1 M.

19. The method of claim 15, wherein the particles comprise silica beads.

20. The method of claim 19, wherein the silica beads are amine functionalized.

21. The method of claim 19, wherein the silica beads have a diameter in a range of 50 nm to 500 nm.
